(19) United States
(12) Patent Application Publication
(10) Pub. No.: US 2025/0186716 A1
(43) Pub. Date: Jun. 12, 2025

(54) MULTI-UNIT DOSE DRY POWDER INHALER AND METHODS OF USE

(71) Applicant: Genentech, Inc., South San Francisco, CA (US)

(72) Inventors: Avi ELIAHU, San Ramon, CA (US); Alice Sylvia HO, Anaheim, CA (US); Alex DRIVER, Cambridge (GB); Julia ALLWRIGHT, Cambridge (GB); Charlie LOWNDES, Cambridge (GB)

(73) Assignee: Genentech, Inc., South San Francisco, CA (US)

(21) Appl. No.: 18/954,361

(22) Filed: Nov. 20, 2024

Related U.S. Application Data
(63) Continuation of application No. PCT/US2023/023053, filed on May 22, 2023.
(60) Provisional application No. 63/365,229, filed on May 24, 2022.

(51) Int. Cl. A61M 15/00

(52) U.S. Cl. CPC A61M 15/0026; A61M 15/0041; A61M 2202/064

(57) ABSTRACT

Exemplary multi-unit dose dry powder inhaler devices may be provided with a housing body having a swirl chamber therein, at least one air inlet, a mouthpiece, a rotatable downfall wheel having at least two capsule spaces that are each configured to hold a capsule, a drive mechanism configured to repeatably index the downfall wheel in a predefined angular increment such that one of the capsule spaces moves adjacent to the swirl chamber each time the downfall wheel is indexed, and a pair of side ramps adjacent to the swirl chamber and the downfall wheel. The side ramps may be configured to cooperate with center ramps located in the capsule spaces to drive a capsule radially outward from a capsule space and into the swirl chamber, thereby enabling the device to automatically load multiple capsules one at a time sequentially into the swirl chamber. Methods of use are also provided.

### CROSS-REFERENCE TO RELATED APPLICATIONS

This application is a continuation of PCT/US2023/023053, filed on May 22, 2023, which claims the benefit of priority to U.S. Provisional Application No. 63/365,229, filed on May 24, 2022, each of which are hereby incorporated by reference herein in their entirety and for all purposes

### INCORPORATION BY REFERENCE

All publications and patent applications mentioned in this specification are herein incorporated by reference for all intents and purposes to the same extent as if each individual publication or patent application was specifically and individually indicated to be incorporated by reference.

### FIELD

Embodiments of the disclosure relate generally to inhaler devices. Specifically, some implementations of the present disclosure relate to dry powder inhaler devices having multi-unit dose capabilities.

### BACKGROUND

The present disclosure relates to inhaler devices, such as for inhaling dry powder medications to treat asthma. Inhaler devices for inhaling the contents of a capsule for medical uses are already known. Available inhalers, however, are not fully satisfactory from an operating standpoint and are susceptible to improvements.

U.S. Pat. No. 7,284,552 to Mauro Citterio, issued on Oct. 23, 2007 and entitled INHALER DEVICE, provides an example of a prior art inhaler device similar to those provided herein. The inhaler device includes an inhaler body defining a recess for a medicine capsule holding a substance to be inhaled, and a nosepiece/mouthpiece communicating with the capsule recess. The device also includes at least one perforating element coupled to the inhaler body and provided for perforating the capsule for allowing an outside airflow to be mixed with the capsule contents and inhaled through the nosepiece/mouthpiece.

U.S. Pat. No. 8,479,730 to Dominik Ziegler et al., issued on Jul. 9, 2013 and entitled INHALER DEVICE, provides another example of a prior art inhaler device. The inhaler device of the U.S. Pat. No. 8,479,730 patent is similar in construction and operation to that of the U.S. Pat. No. 7,284,552 patent, but has a mouthpiece that is pivotally attached to an edge of the inhaler body.

The above inhalers are single dose devices that require the user to perform many steps to administer each dose. What is needed and not provided by prior art inhalers are devices that require fewer steps and are easier to use.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 is an exploded perspective view of an exemplary embodiment of an inhaler device according to the present disclosure;

FIG. 2 is a further perspective view of the exemplary inhaler device shown in an open condition thereof, i.e., in the capsule loading position thereof;

FIG. 3 is a view similar to FIG. 2, but illustrating the inhaler device according to the present disclosure during the use thereof;

FIG. 4 is an elevation cross-sectional view of the inhaler device, shown with a capsule arranged therein, but in a non-perforated condition;

FIG. 5 is a view similar to FIG. 4, but illustrating the inhaler device according to the present disclosure during the capsule perforating operation;

FIG. 6 is a top plan view, as partially cross-sectioned, of the inhaler device according to the present disclosure;

FIG. 7 is a perspective cross-sectional view of the inhaler device illustrating airflow through the device;

FIG. 8 is a perspective view of an exemplary multi-unit dose dry powder inhaler device constructed according to aspects of the present disclosure;

FIG. 9 is a front side view of the inhaler device of FIG. 8;

FIG. 10 is a rear side view of the inhaler device of FIG. 8;

FIG. 11 is a left side view of the inhaler device of FIG. 8;

FIG. 12 is a right side view of the inhaler device of FIG. 8;

FIG. 13 is a top view of the inhaler device of FIG. 8;

FIG. 14 is a bottom view of the inhaler device of FIG. 8;

FIGS. 15-1 through 15-6 are a series of front side views schematically showing operational steps of the inhaler device of FIG. 8;

FIG. 16 is an exploded view of the inhaler device of FIG. 8;

FIG. 17 is a rear side cross-sectional view of the inhaler device of FIG. 8;

FIG. 18 is a front side perspective view of the chassis of the inhaler device of FIG. 8;

FIG. 19 is a front side view of the chassis of the inhaler device of FIG. 8;

FIG. 20 is a rear side view of the chassis of the inhaler device of FIG. 8;

FIG. 21 is a rear side view of the front chassis cover of the inhaler device of FIG. 8;

FIG. 22 is a front side perspective view of the carousel of the inhaler device of FIG. 8;

FIG. 23 is a rear side perspective view of the carousel of the inhaler device of FIG. 8;

FIG. 24 is a front side perspective view of the downfall wheel of the inhaler device of FIG. 8;

FIG. 25 is a rear side perspective view of the downfall wheel of the inhaler device of FIG. 8;

FIG. 26 is a front side view of internal components of the inhaler device of FIG. 8;

FIGS. 27A-27O are a series of rear side views of the chassis and internal components of the inhaler device of FIG. 8;

FIG. 27P is a side view showing a representative medicine capsule after both hemispherical ends have been automatically pierced according to aspects of the disclosure; and

FIGS. 28A-46D are various views of the interlocking latch components of the inhaler device of FIG. 8.

### DETAILED DESCRIPTION

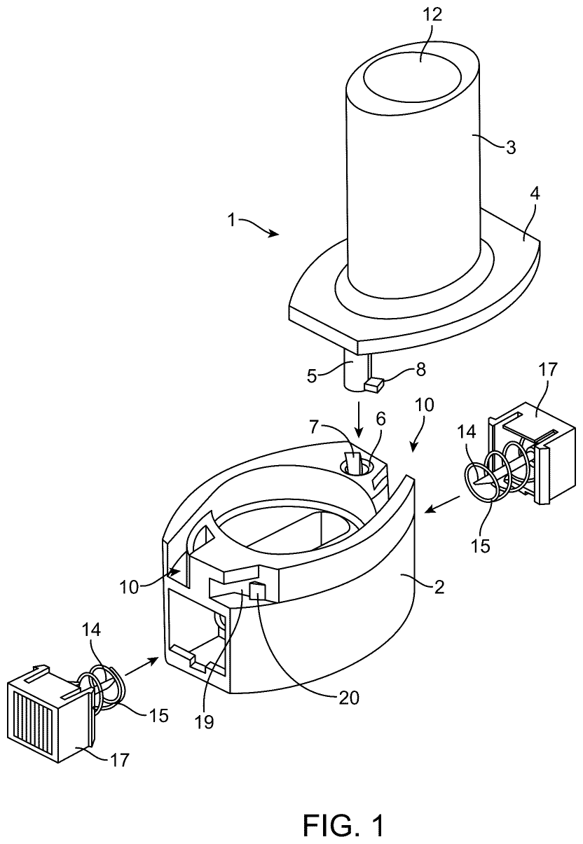

Referring to the reference numerals of the above-mentioned figures, an exemplary single dose inhaler device 1 is described below. As best seen in FIG. 1, the exemplary inhaler device 1 comprises an inhaler mouthpiece 3, including a flange 4, having a peg 5 which can be engaged in a corresponding hole 6 formed in an inhaler body 2. While the term “mouthpiece” is used herein, it is to be understood that in some embodiments this feature may be used as a mouthpiece and or a nosepiece.

The hole 6 is provided with a longitudinal slot (not shown), that can engage a cross tooth 8 of the peg 5, and a bottom ring-like recess, not specifically shown, in which the tooth 8 can slide.

Thus, it is possible to engage the peg 5 in the hole, by causing the tooth 8 to pass through the slot 7 and, upon achieving the bottom, it is possible to fully rotate the peg 5 in its hole 6, thereby also rotating the inhaler mouthpiece 3 with respect to the inhaler body 2.

The inhaler mouthpiece 3 can be locked in its closed condition, shown in FIGS. 3-6, by a snap type of locking means, including a hook portion 18 of the flange 4 having a small ridge, not shown, for engaging a corresponding ridge 20 formed inside a latching recess 19, defined in the inhaler body 2.

The inhaler body 2 is moreover provided with a recess for the capsule, the recess being upward opened and communicating with the outside through a perforated plate or grid 11, included in the inhaler mouthpiece 3 at the flange 4 and designed for separating the capsule recess 9 from the duct 12 of the mouthpiece.

A capsule 13 can be engaged in the recess 9, the capsule being of a per se known type and adapted to be perforated to allow the drug contents held therein to be easily accessed, the perforating operation being performed by any suitable perforating means.

In the disclosed embodiment, the perforating means comprise a pair of perforating needles 14 which can transversely slide as counter-urged by resilient elements comprising, in this embodiment, coil springs 15; each coil spring coaxially encompassing the perforating needle 14 and operating between a respective abutment element 16, rigid with the inhaler body 2, and a hollow push-button element 17. The perforating needles 14 may be similar to hollow hypodermic needles and have a single-side beveled tip, for facilitating the perforating needles 14 in perforating the coating of the capsule 13. In other implementations, the perforating needles 14 may be solid and or have other tip configurations.

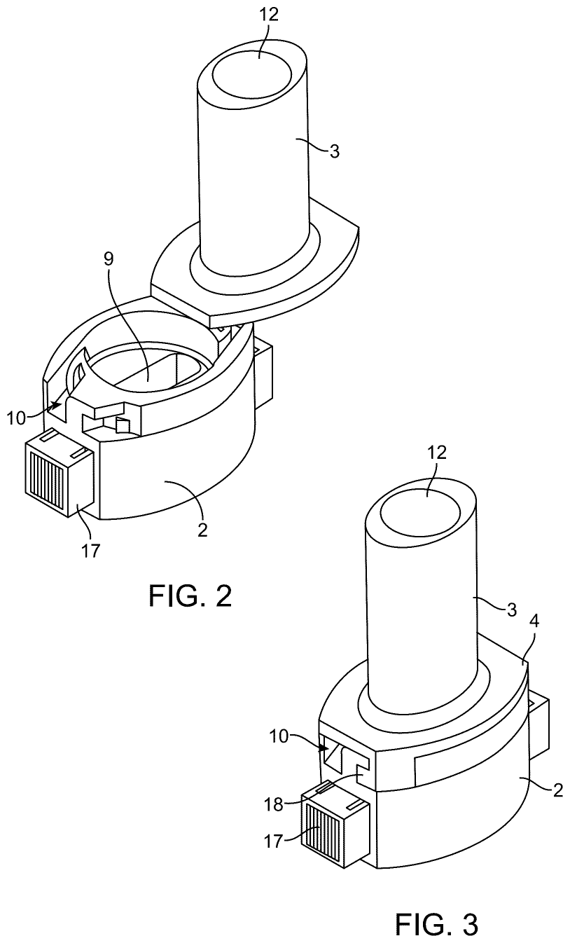

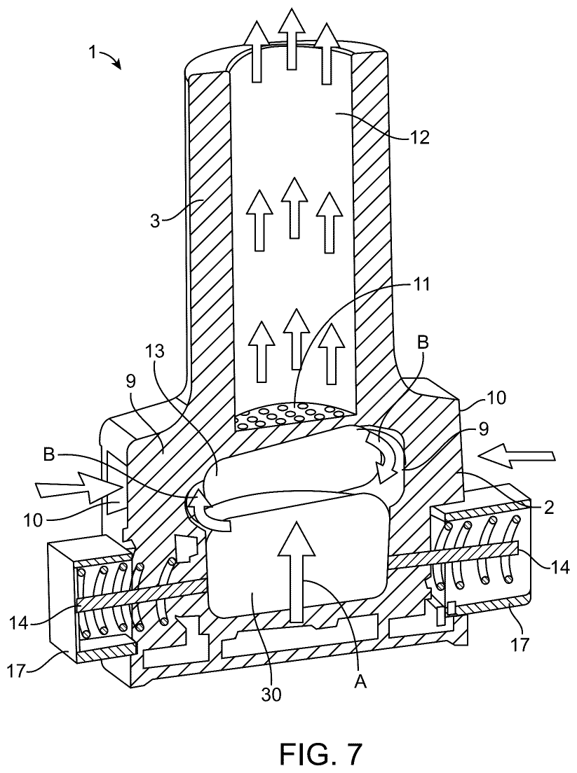

The operation of the inhaler device according to the present disclosure is as follows. In the open condition, as shown in FIG. 2, a capsule is engaged in the capsule recess 9 and the mouthpiece 3 is snapped closed on the inhaler body 2. By pressing the push-button elements 17, the perforating needles 14 will perforate the capsule 13, thereby its contents, usually a fine powder, will be communicated with the capsule recess. By applying suction on the mouthpiece 3, an airflow is generated which, coming from the outside through the inlets 10, will enter the capsule recess, thereby mixing with the capsule contents. The tangential orientation of inlets 10 relative to the capsule recess 9 causes the incoming air to generate a swirling airflow. This swirling airflow lifts capsule 13 upward (shown by arrow A in FIG. 7) out of capsule pocket 30 and into the larger, upper portion of capsule recess 9. The swirling airflow further spins capsule 13 within recess 9 as shown by arrows B, generally about a transverse axis of the capsule and generally about the longitudinal axis of recess 9 (i.e., a generally vertical axis in FIG. 7). However, since the diameter of recess 9 is larger than the length of capsule 13, the capsule may travel around recess 9 as it spins rather than spinning around a single fixed axis. Centrifugal force from the spinning capsule 13 assists its contents in exiting the pierced ends of the capsule, where it is aerosolized by the swirling airflow, passes through mouthpiece grid 11 and duct 12, and is inhaled by the user. In some embodiments, dry powder deagglomeration is achieved by: 1) shear through the pierced holes in the capsule; 2) turbulence from swirling airflow in the capsule chamber; and 3) particle collisions (with the walls of the device, with the mouthpiece grid, and with other particles.)

Inhaler device 1 has a very simple construction. A further advantage of inhaler device 1 is the specifically designed configuration of the perforating needles that can be assimilated, as stated, to hypodermic needles. Since this type of needle presents a very small resistance against perforation and a very accurate operation, it is possible to use needles having a comparatively large diameter, without damaging the capsule, thereby providing a very simple perforating operation. The use of a small number of perforating needles, only two in some embodiments, allows reducing the contact surface between the needle and capsule (the perforated cross section being the same), with a consequent reduction of friction and of the problems affecting the prior inhalers.

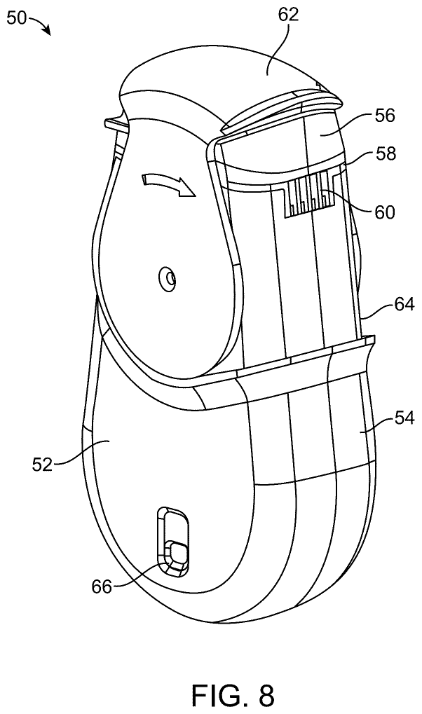

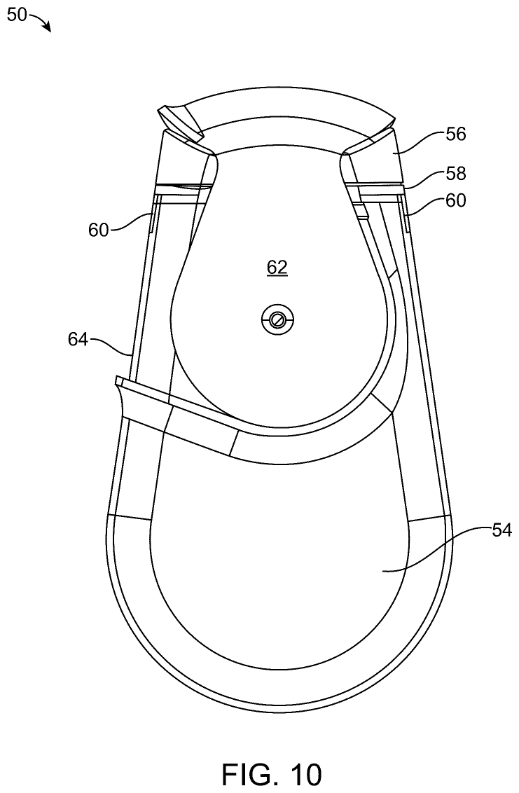

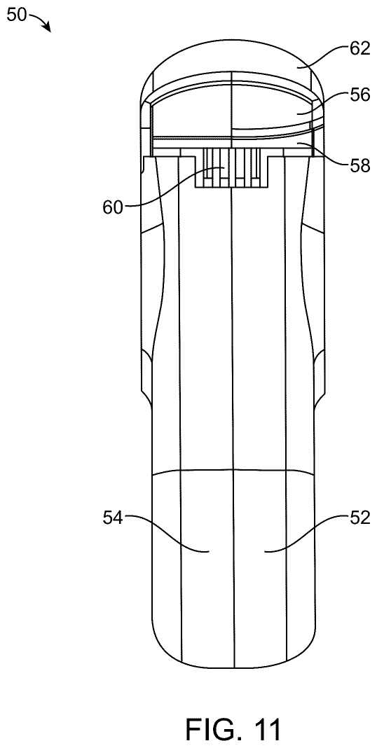

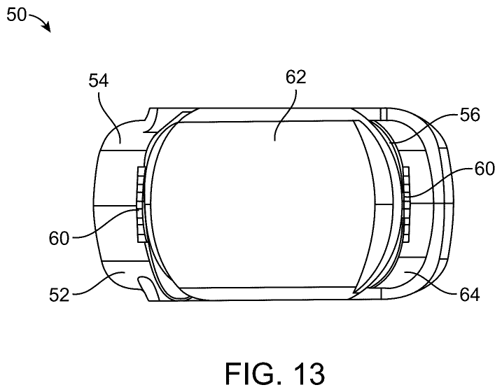

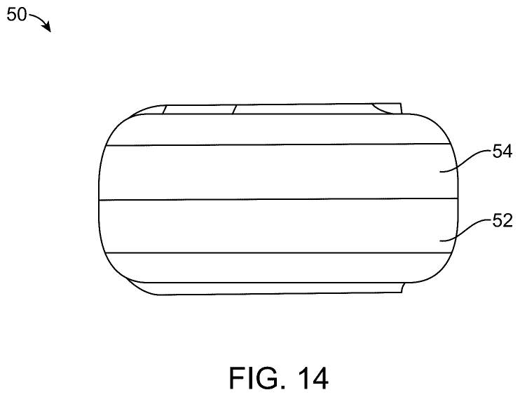

Referring to FIGS. 8-14, another exemplary inhaler device 50 constructed according to aspects of the present disclosure is shown. Device 50 is a multi-unit dose dry powder inhaler. It includes a capsule recess or swirl chamber similar in construction and operation to that of previously described inhaler device 1. However, instead of requiring a user to manually insert one medicine capsule at a time, device 50 is provided to a user pre-loaded with multiple capsules which are sequentially loaded into the swirl chamber automatically. In this exemplary embodiment, device 50 may be provided with up to 30 preloaded capsules. Because inhaler device 50 may be used with individual capsules having a standard size rather than medicine packaged in compartments of a blister strip, standard capsule filling equipment may be used to provide various formulations of medicine for the device rather than requiring special blister pack filling equipment. Another advantage of capsules over blisters is that they can contain larger volumes of powder, which enables higher dose delivery per inhalation.

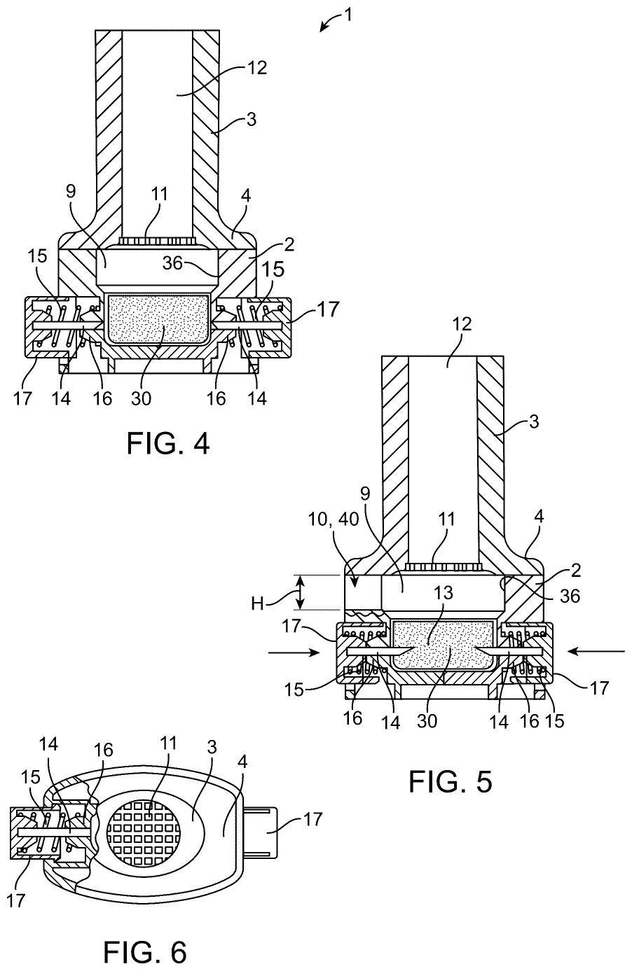

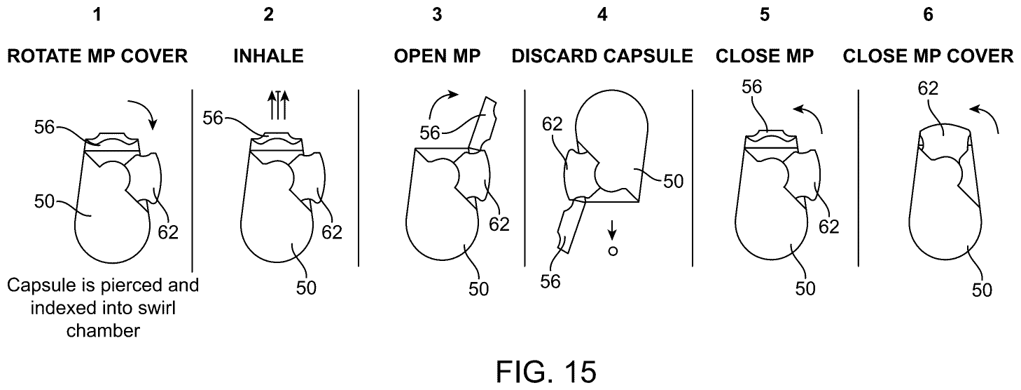

In this exemplary embodiment, inhaler device 50 is generally pear or teardrop shaped and has a main body formed by a front cover 52 and a rear cover 54. Covers 52 and 54 may be assembled with fasteners, plastic snap features, adhesives, ultrasonic welding, and/or other suitable assembly methods. A mouthpiece 56 may be provided above a capsule chamber portion 58. Mouthpiece 56 may be hingedly attached to capsule chamber portion 58 such that it pivots about a horizontal pivot axis between a closed position (as shown) and an open position (see FIG. 15-3) in which an empty medicine capsule may be removed from capsule chamber portion 58 (see FIG. 15-4.) A recess 106 (see FIG. 18) may be formed in capsule chamber portion 58 similar to recess 9 shown in FIG. 4, as previously described. In some embodiments, the main portion of swirl chamber/capsule recess 106 is less than 0.26 inches tall, or less than 12% taller than the diameter of the capsules it receives. This recess is shallower than prior art devices and is believed to reduce powder deposition and improve delivery efficiency. Air channels (see FIG. 18) may be provided on opposite sides of the recess in fluid communication with air vents 60 to provide intake air for swirling a medicine capsule within the recess, as previously described. In some embodiments, swirl chamber/capsule recess 106 is not configured to enable a capsule to spin, but only directs one or more air flows over, around and or through the capsule.

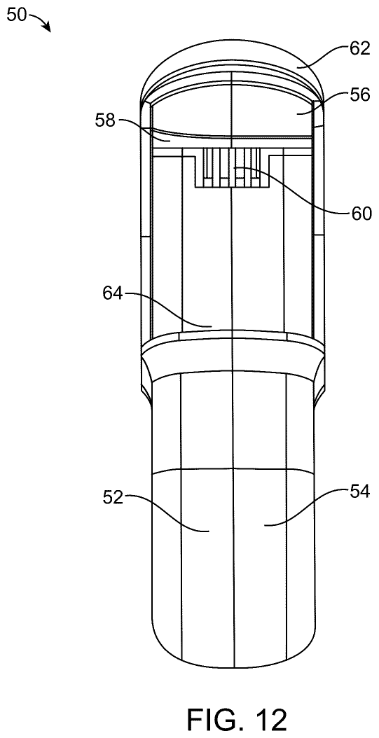

A pivoting mouthpiece cover 62 may be provided over mouthpiece 56. In this exemplary embodiment, mouthpiece cover 62 extends over mouthpiece 56 and includes a pair of downwardly depending arms that extend over a top portion of the front cover 52 and the rear cover 54. Mouthpiece cover 62 pivots about a horizontal axis that extends between its two arms and moves between a closed position (as shown) through about 90 degrees to an open position in which mouthpiece 56 is exposed (see FIG. 15-1.) In other embodiments, this angular range of motion can be larger or smaller, depending on the function of internal mechanisms driven by opening and closing a mouthpiece cover. As best seen in FIGS. 8 and 12, a recess 64 may be provided in front cover 52 and rear cover 54 to at least partially receive mouthpiece cover 62 and provide a stop for when cover 62 is being opened. In this exemplary embodiment, device 50 is 30 mm thick across the front and rear covers, and has a maximum thickness of less than 35 mm across the downwardly depending arms of mouthpiece cover 62. In this exemplary embodiment, device 50 is less than 120 mm tall and less than 65 mm wide at its widest point.

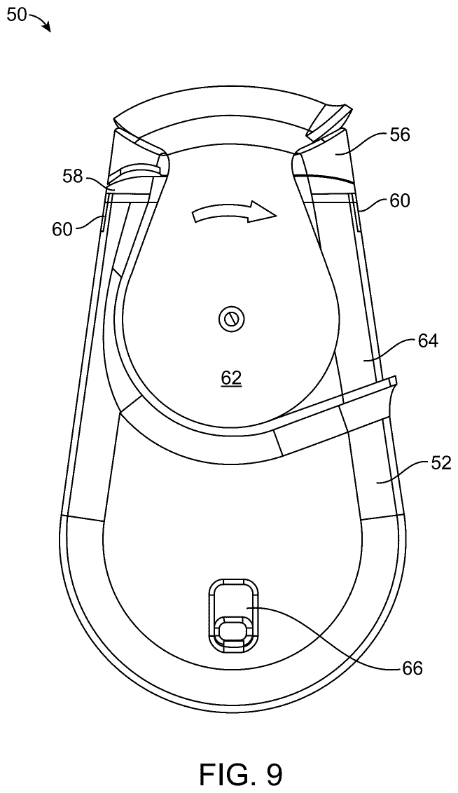

A dose count aperture 66 may be provided in front cover 52 for indicating to the user how many doses remain before device 50 is depleted, as shown in FIGS. 8 and 9.

Referring to FIGS. 15-1 through 15-6, a series of steps are schematically depicted, illustrating the overall operation of inhaler device 50. In this exemplary embodiment, the user first rotates mouthpiece cover 62 from a closed position to an open position, as depicted in FIG. 15-1, where it clicks into place. This action uncovers mouthpiece 56, pierces one of the medicine capsules preloaded into device 50 and indexes it into the swirl chamber. This action also advances a dose count wheel so that the dose count showing through the dose count aperture is decremented by one.

After mouthpiece cover 62 is rotated into the open position, the user places mouthpiece 56 against their mouth and inhales the dry powder released from the swirling capsule, as depicted in FIG. 15-2 and as previously described. The user then flips open mouthpiece 56 as depicted in FIG. 15-3, inverts device 50 to discard the empty medicine capsule from the swirl chamber as depicted in FIG. 15-4, and closes mouthpiece 56 as depicted in FIG. 15-5. Finally, the user closes mouthpiece cover 62 as depicted in FIG. 15-6, readying device 50 for repeating the above procedure when the next dose is to be inhaled. In some embodiments, mouthpiece cover 62 cannot be moved to the closed position (or will not remain in the closed position) unless mouthpiece 56 has been opened and closed first, as will be subsequently described in more detail.

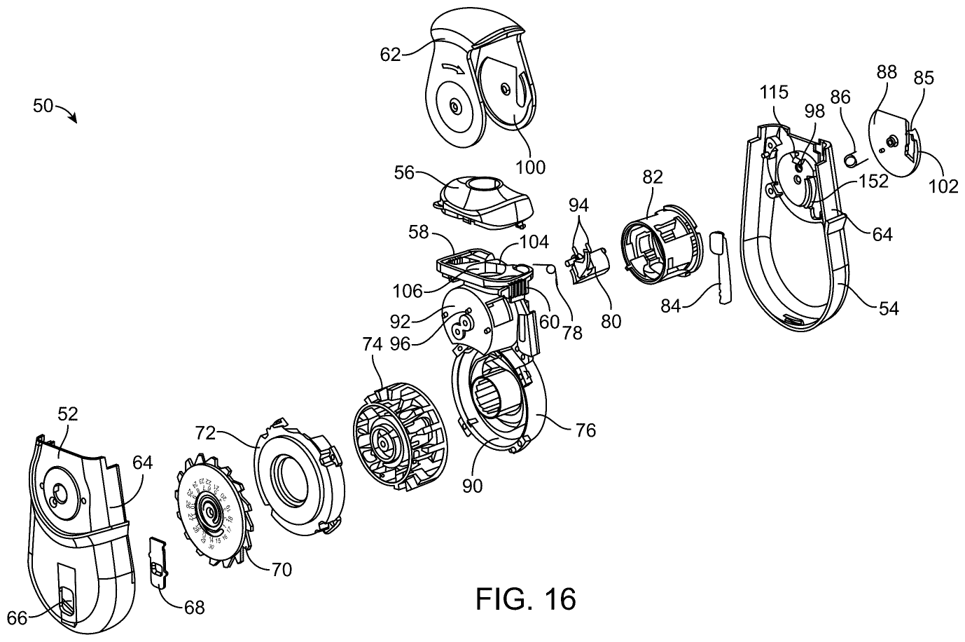

Referring to FIG. 16, an exploded perspective view shows the components of exemplary inhaler device 50 (with fasteners omitted for clarity.) Device 50 includes (shown from left to right) front cover 52, sliding dose count window 68, dose count wheel 70, front chassis cover 72, carousel 74, chassis 76 with capsule chamber portion 58, mouthpiece 56, mouthpiece cover 62, hub spring 78, piercing hub 80, downfall wheel 82, latch arm 84, rear cover 54, mouthpiece cover spring 86 and drive plate 88.

When device 50 is assembled, carousel 74 is rotatably received in a forward-facing lower cavity 90 of chassis 76. Carousel 74 is captivated in lower cavity 90 by front chassis cover 72, which may be secured to chassis 76 by four fasteners (not shown), or any means suitable for registration and attachment. A central hub of carousel 74 extends in a forward direction through a central aperture in front chassis cover 72 so that it can engage with dose count wheel 70. Dose count wheel 70 may be attached to carousel 74 with a single fastener through its center or any means suitable for registration and attachment so that it can rotate with carousel 74. A pair of forwardly protruding pegs or other registration features may be provided on the central hub of carousel 74 for engaging with mating recesses in the back side of dose count wheel 70 to ensure that the dose count wheel stays properly indexed with carousel 74. Sliding dose count window 68 is received within a mating slot on the inside/backside of front cover 52 which sandwiches sliding window 68 between cover 52 and wheel 70. This arrangement allows sliding window 68 to slide up and down, as will be subsequently described in more detail. Front cover 52 may be secured to the front side of chassis 76 with a single fastener (not shown), and or any other means suitable for registration and attachment.

Downfall wheel 82 is configured to be rotatably received in a rearward facing upper cavity 92 of chassis 76. Downfall wheel is captivated in upper cavity 92 by rear cover 54, which may be secured to the backside of chassis 76 by three fasteners (not shown), and or any other means suitable for registration and attachment.

Piercing hub 80 may be provided with two piercing pins or sharps 94 configured to pierce the same side of a medicine capsule on opposite hemispherical ends of the capsule (perpendicular to the longitudinal axis of the capsule), rather than piercing the capsule through its ends along the longitudinal axis as done by the previously described device 1. When device 50 is assembled, piercing hub 80 resides in the central bore of downfall wheel 82. Hub 80 has a forward extending axle that is received in mating bore 96 in upper cavity 92 of chassis 76, and a rearward extending axle that is received in mating bore 98 in the inside surface of rear cover 54. With this arrangement, piercing hub 80 is configured to pivot about a front to back horizontal axis allowing piercing pins to rotate between a lower position and an upper position, as will be subsequently described in more detail. Torsion hub spring 78 may be provided between hub 80 and chassis 76 to bias piercing pins 94 towards the lower position. In other embodiments (not shown), a four-bar mechanism may be used instead of a rotating piercing hub to move piercing pins 94 into the capsules.

Latch arm 84 may be configured to slide up and down in a vertical channel formed between the backside of chassis 76 and the inside of rear cover 54. Latch arm 84 serves to prevent mouthpiece cover 62 from returning to or staying in its upper position covering mouthpiece 56 until after mouthpiece 56 has been opened and closed, as previously described. Details of the construction and operation of latch arm 84 will be subsequently described.

Drive plate 88 may be configured to reside in a mating recess 100 in the rear arm of mouthpiece cover 62, such that drive plate 88 rotates with cover 62. Drive plate 88 includes a circumferentially extending flexure arm 102 and tooth 85 that extends through an arcuate slot in rear cover 54 to allow mouthpiece cover 62 to rotationally drive downfall wheel 82 ninety degrees at a time, as will be subsequently described in detail. Mouthpiece cover spring 86 may be provided between drive plate 88 and rear cover 54 to bias mouthpiece cover 62 towards its lower/open state. In some embodiments, features of drive plate 88 may be provided directly into mouthpiece cover 62 and the drive plate itself may be omitted.

Mouthpiece 56 may be provided with hinge features along its rear edge (not shown) for mating with hinge features 104 on the rear top edge of chassis 76. This arrangement allows mouthpiece 56 to pivot about a longitudinal horizontal axis between a closed position and an open position.

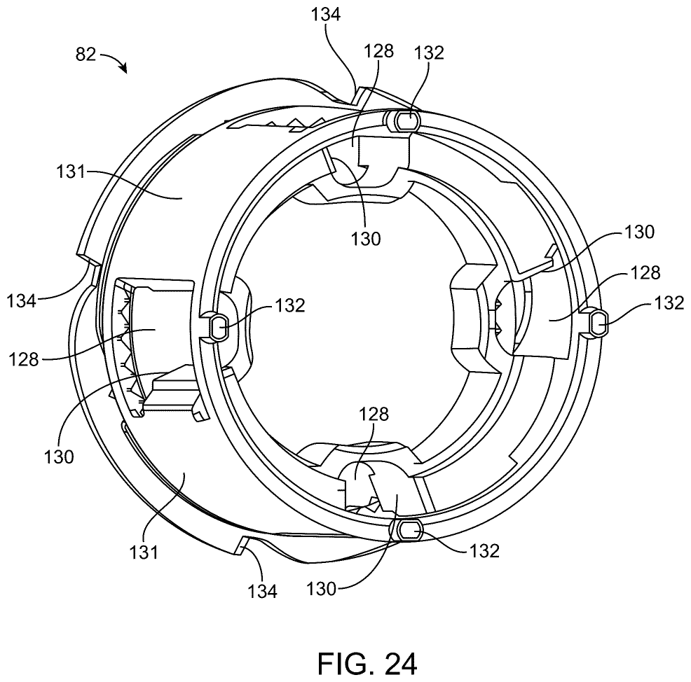

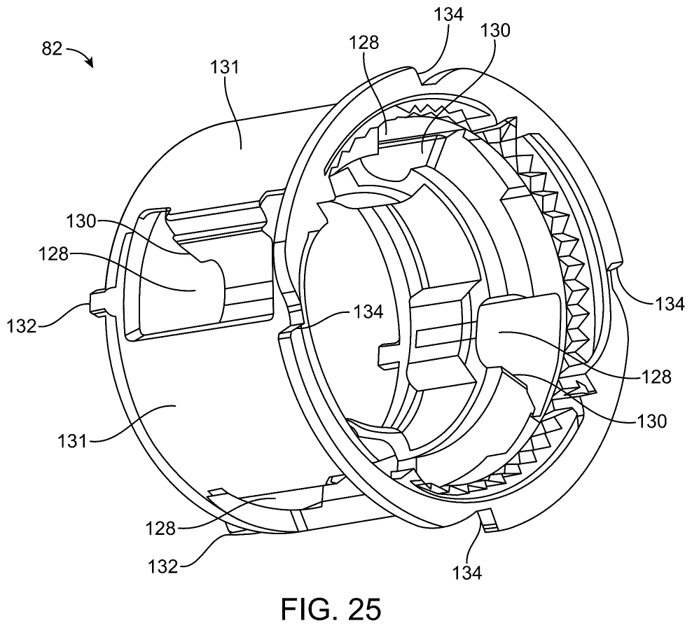

Rear cover 54 may be provided with an inwardly facing upper ramp 115, as shown. The function of ramp 115 is described below in the discussion of FIGS. 24 and 25.

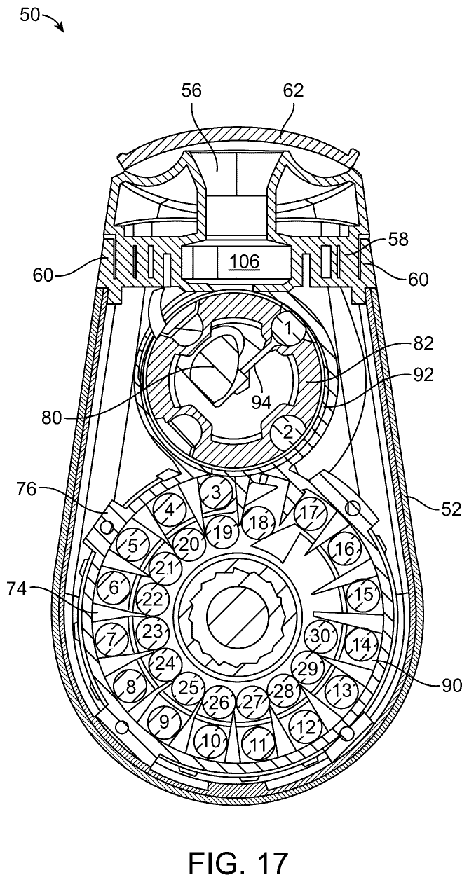

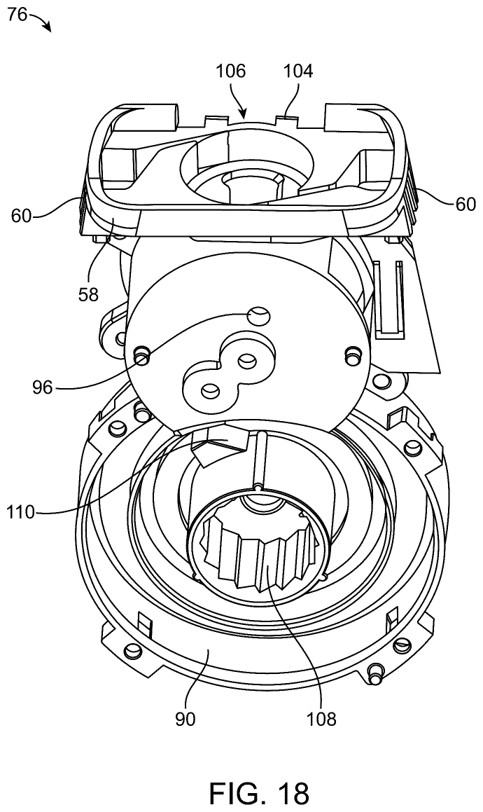

Referring to FIG. 17, the overall capsule indexing sequence of inhaler device 50 will be described. In this exemplary embodiment, device 50 is configured to be pre-loaded with 30 medicine capsules. In FIG. 17, the capsules are numbered from 1 to 30, with number 1 being the first dose and number 30 being the last dose. Capsules 1 and 2 are loaded into two of the four spaces that are located 90 degrees apart around the circumference of downfall wheel 82. Capsules 3-17 are loaded into 15 spaces that are located 22.5 degrees apart forming an outer ring around the circumference of carousel 74. Capsules 18-30 are loaded into 13 spaces that are also located 22.5 degrees apart forming an inner ring within carousel 74. Alternatively, capsules 1-15 are loaded into 15 outer ring spaces and capsules 16-30 are loaded into 15 inner ring spaces. In the some embodiments, all of the capsules can be loaded from the same side of the device (from the front side of carousel in this exemplary embodiment.) The indexing mechanism is then activated twice to index capsules 1 and 2 from the carousel outer ring into downfall wheel positions 1 and 2 to prepare the device ready for use. This leaves two empty spaces in the inner ring, as shown. In other embodiments, a downfall wheel may have fewer or more than 4 capsule spaces. In some embodiments, the carousel can be omitted and means provided for the user to insert capsules into the downfall wheel from the side of the device to provide a mono-dose or multi-dose device with an auto-pierce solution.

During operation, downfall wheel 82 rotates counterclockwise (as viewed from the rear of device 50) and carousel 74 rotates clockwise. Downfall wheel 82 indexes 90 degrees at a time and carousel 74 indexes 22.5 degrees. Drive plate 88 (FIG. 16), which is coupled to the inside of mouthpiece cover 62 (FIG. 16), drives downfall wheel 82 (FIG. 17) each time cover 62 is moved 90 degrees from the closed position to the open position, as will be subsequently described in more detail. Each time downfall wheel 82 is indexed 90 degrees, it drives the carousel 22.5 degrees through a Geneva-type mechanism (not shown), as will also be subsequently described in more detail. With this arrangement, when mouthpiece cover 62 is opened, the capsule in the number 1 position is pierced and moved into swirl chamber 106, and each remaining capsule is advanced into the position previously occupied by the preceding capsule. In other words, capsule number 2 stays in its cavity but is advanced into the position previously occupied by capsule number 1, capsule number 3 is advanced from carousel 74 into the position (but in a different cavity) of downfall wheel 82 previously occupied by capsule number 2, and so on. This means that capsule number 30 eventually advances through each of the positions in the inner ring of carousel 74, is moved into the outer ring of carousel 74, advances through each of the positions in the outer ring, is moved from carousel 74 into position number 2 of downfall wheel 82 and is advanced into position number 1 before it is loaded into swirl chamber 106.

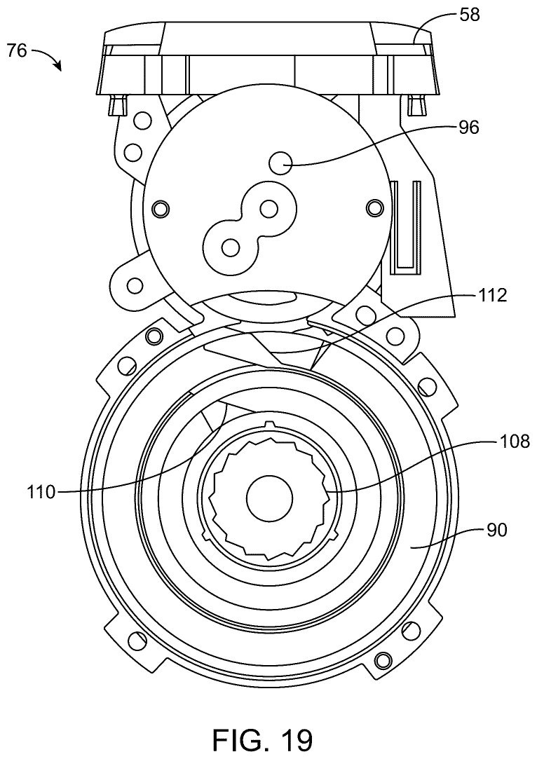

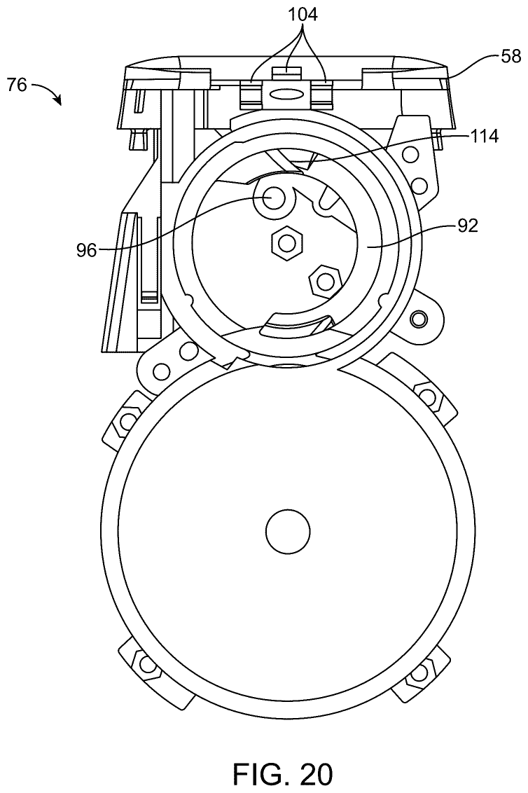

Referring to FIG. 18-20, details of chassis 76 are shown. As shown in FIGS. 18 and 19, the central hub of lower cavity 90 is provided with a series of ratchet teeth 108 along its inside surface to prevent carousel 74 (not shown) from rotating in a reverse direction. As best seen in FIG. 19, lower cavity 90 may be provided with an inner ramp 110 configured to guide the rearward ends of the medicine capsules upwardly from the inner ring to the outer ring of the chassis cavity 90 of chassis 76 as they are being advanced by the rotation of carousel 74 (shown in FIG. 17.) Lower cavity 90 may also be provided with an outer ramp 112 configured to guide the rearward ends of the medicine capsules upwardly from the outer ring of chassis cavity 90 of chassis 76 into the upper cavity 92 of chassis 76 by the rotation of carousel 74, and into the downfall wheel 82 (shown in FIG. 17.) As shown in FIG. 20, upper cavity 92 may be provided with an upper ramp 114 configured to guide the forward ends of the medicine capsules upwardly as they are being advanced from the downfall wheel and into swirl chamber 106.

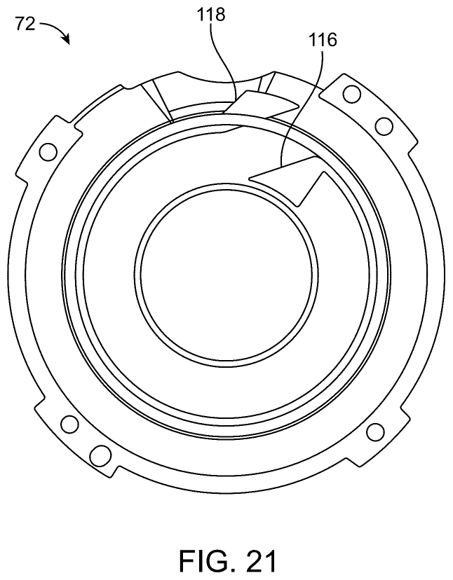

Referring to FIG. 21, details of front chassis cover 72 are shown. Cover 72 may be provided with an inner ramp 116 (corresponding with inner ramp 110 shown in FIG. 19) configured to guide the forward ends of the medicine capsules upwardly from the inner ring to the outer ring of chassis cover 72 as they are being advanced by the rotation of carousel 74 (shown in FIG. 17.) Cover 72 may also be provided with an outer ramp 118 (corresponding with outer ramp 112 shown in FIG. 19) configured to guide the forward ends of the medicine capsules upwardly from the outer ring of chassis cover 72 into the upper cavity 92 of chassis 76 by the rotation of carousel 74, and into the downfall wheel 82 (shown in FIG. 17.)

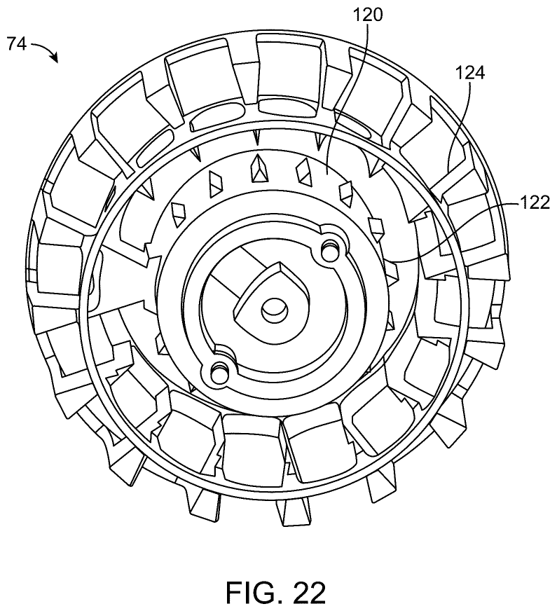

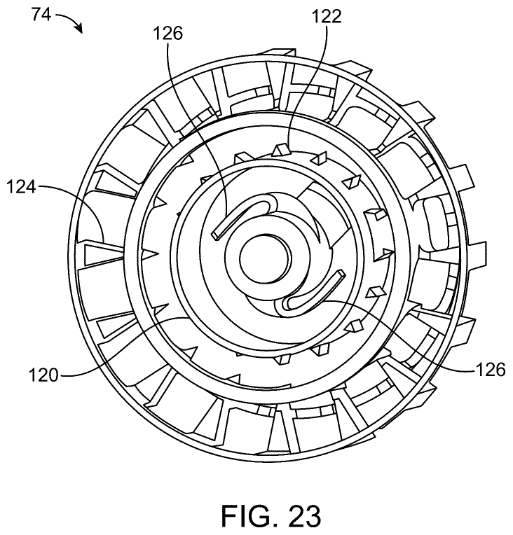

Referring to FIGS. 22 and 23, details of carousel 74 are shown. The outer circumference of central hub 120 may be provided with triangular protrusions 122 between the capsule spaces. Central ramps 124 may be provided as shown, having pushing or driving surfaces configured to work in cooperation with triangular protrusions 122 and the previously described side ramps to move the capsules from the inner ring to the outer ring, and also from the outer ring to the upper cavity 92/downfall wheel 82. In this embodiment, center ramps 124 have a width that is about 40% of the length of a capsule, and the previously described side ramps each have a width that is about 30% of the capsule. In some embodiments, the side ramps have a width that is at least 27%, 28%, 29% 30%, 31%, 32 or 33% the length of a capsule. In other embodiments (not shown), ramp locations may be swapped by providing stationary center ramps on a chassis, and side ramps provided on a carousel and or a downfall wheel. As shown in FIG. 23, one or more flexure arms 126 may be provided inside central hub 120. Flexure arms 126 can be configured to work in cooperation with ratchet teeth 108 (shown in FIGS. 18 and 19) to prevent carousel 74 from rotating in a reverse direction.

Referring to FIGS. 24 and 25, details of downfall wheel 82 are shown. As previously described, downfall wheel 82 may be provided with four capsule spaces 128, only two of which are occupied at any one time. Each capsule space 128 may be provided with a center ramp 130 for working in cooperation with the previously described upper side ramps 114 and 115 to move each capsule in turn from upper cavity 92 of chassis 76/downfall wheel 82 into the swirl chamber (not shown.) In this embodiment, spaces are left between center ramp 130 and the side ramps so that piercing pins 94 can pass between the center ramp and side ramps. In this exemplary embodiment, piercing pins 94 have a diameter of about 0.047 inches. Upper side ramps 114 and 115 (shown in FIGS. 20 and 16, respectively) are configured to engage the hemispherical ends of the capsules while center ramp 130 is configured to engage the center cylindrical portion of the capsules. As such, ramps 114 and 115 may be provided with compound angles (i.e., angled with respect to a vertical plane transverse to the rear cover and also angled with respect to a horizontal plane. In some embodiments, ramps 114 and 115 are angled about 45 degrees with respect to both of these planes. In some embodiments, ramps 114 and 115 have an angle between 30 and 60 degrees with respect to both of these planes.

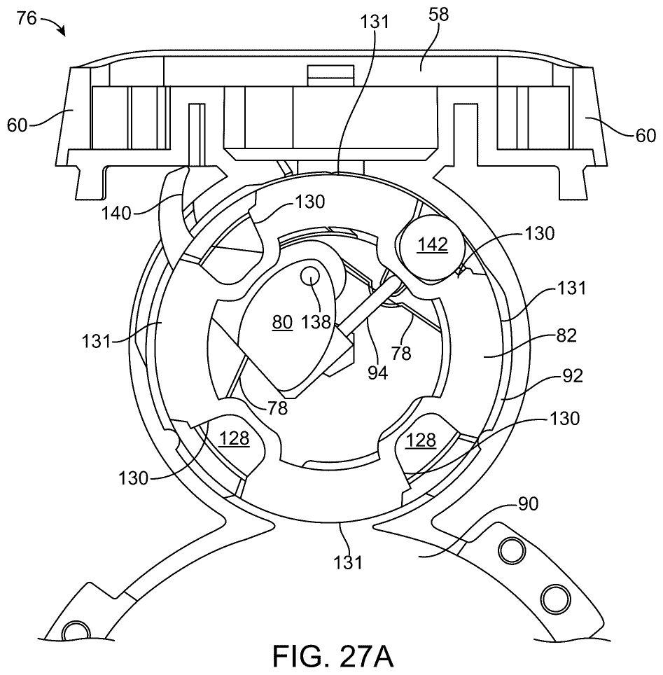

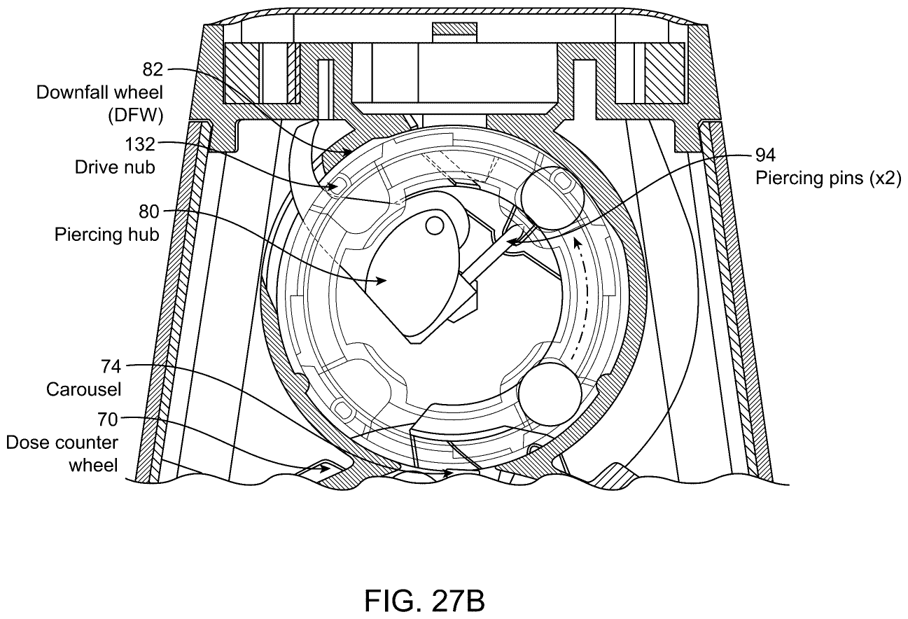

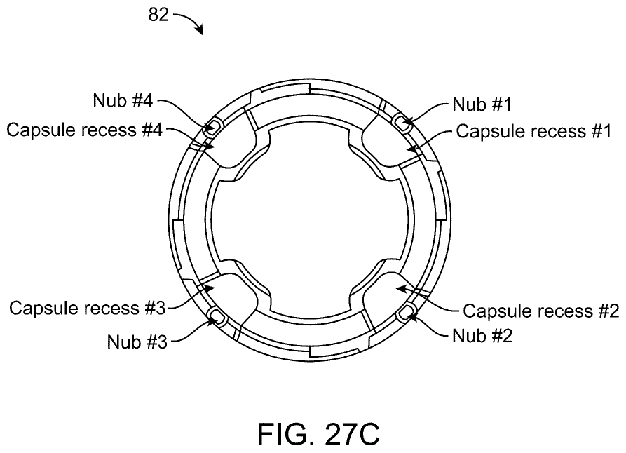

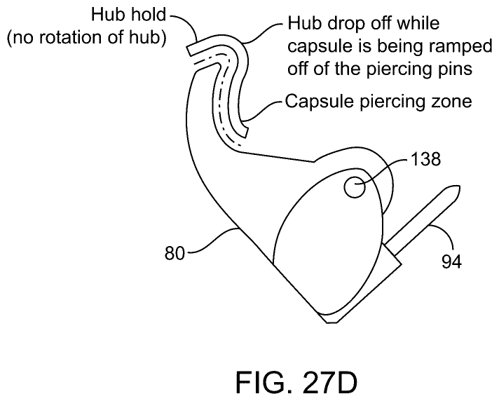

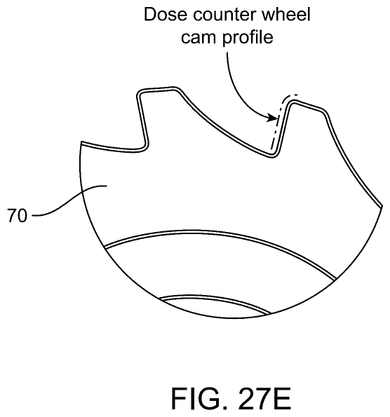

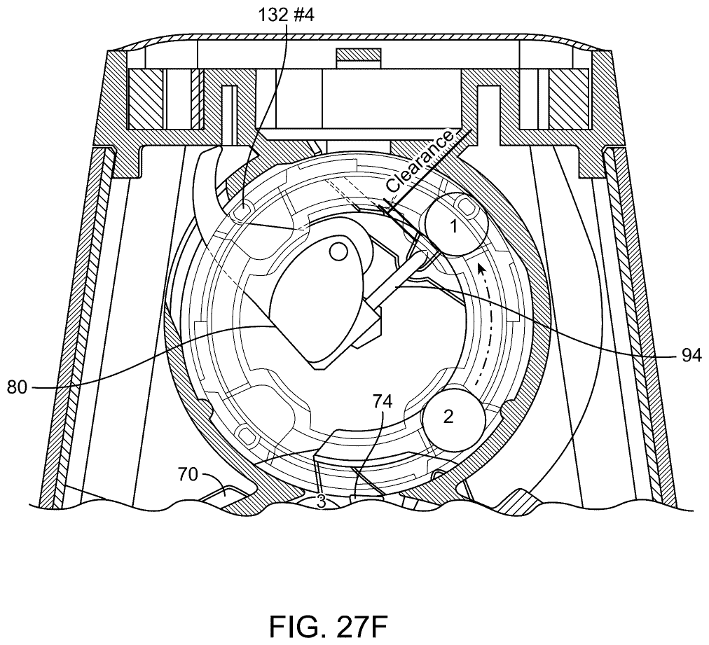

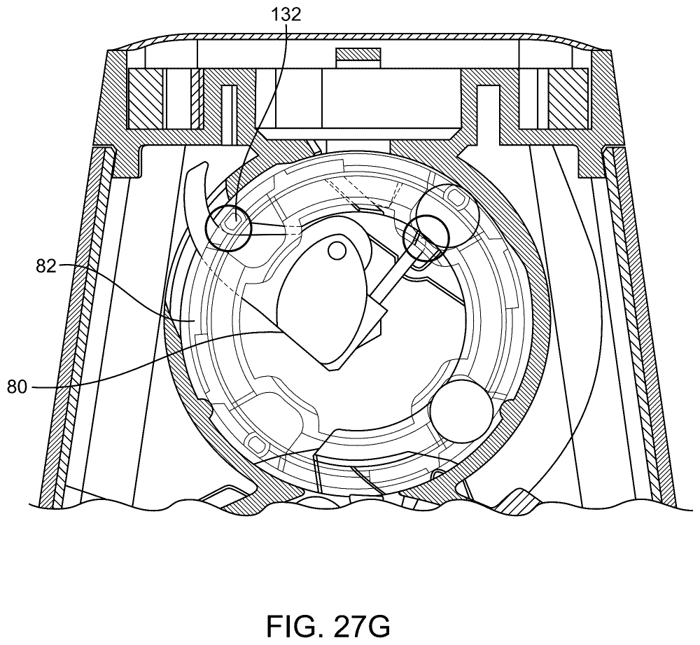

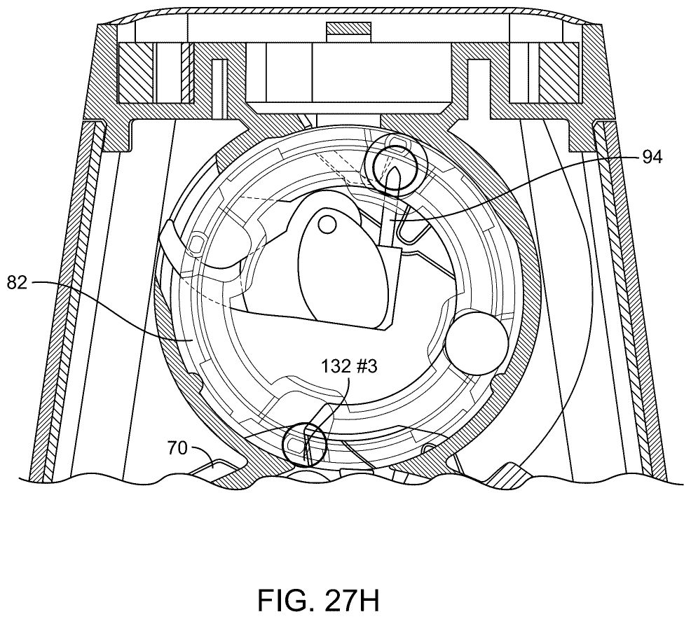

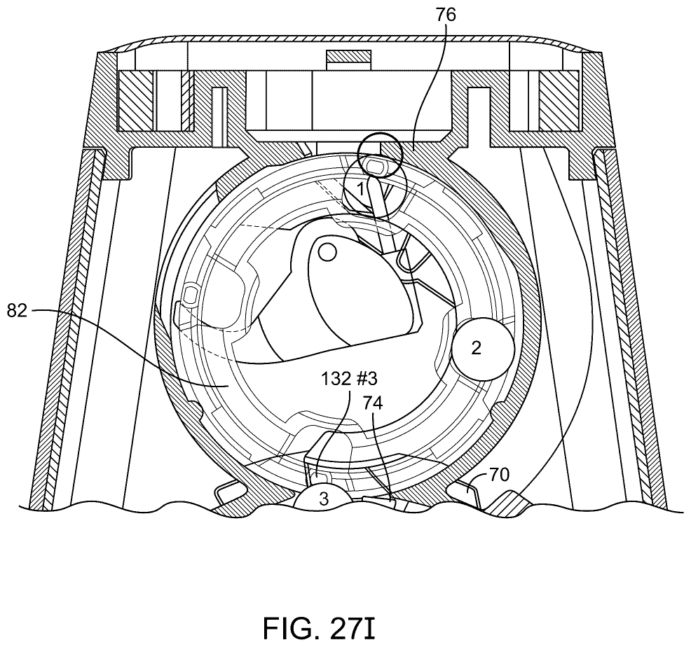

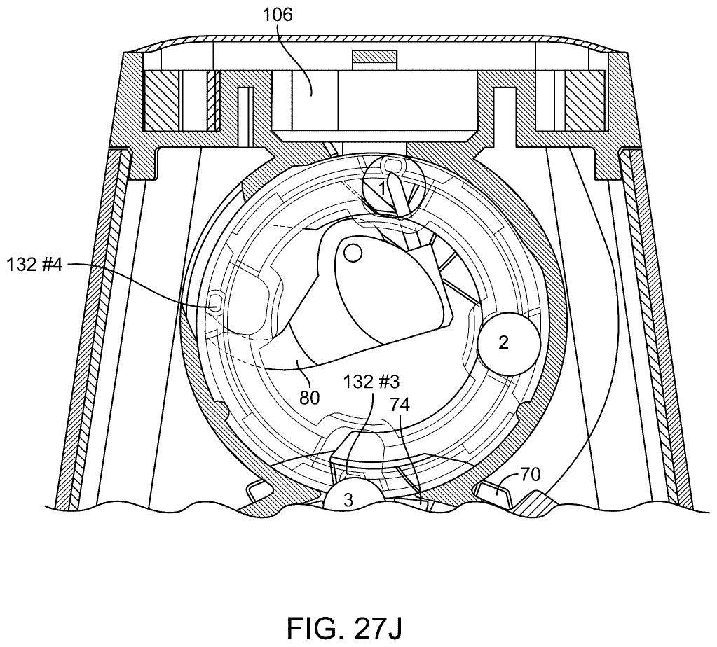

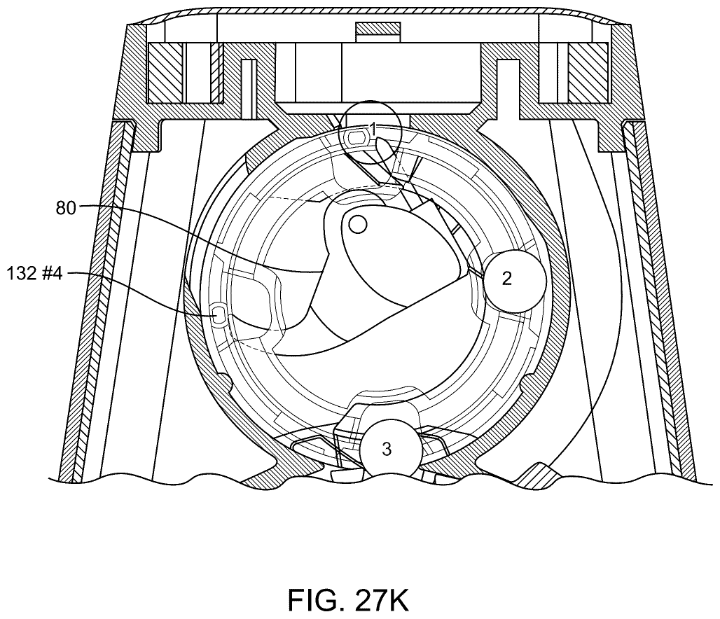

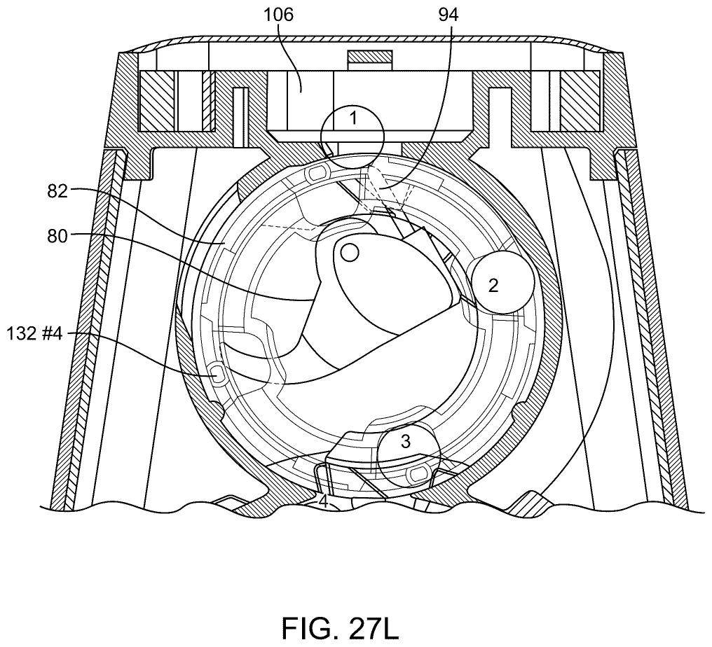

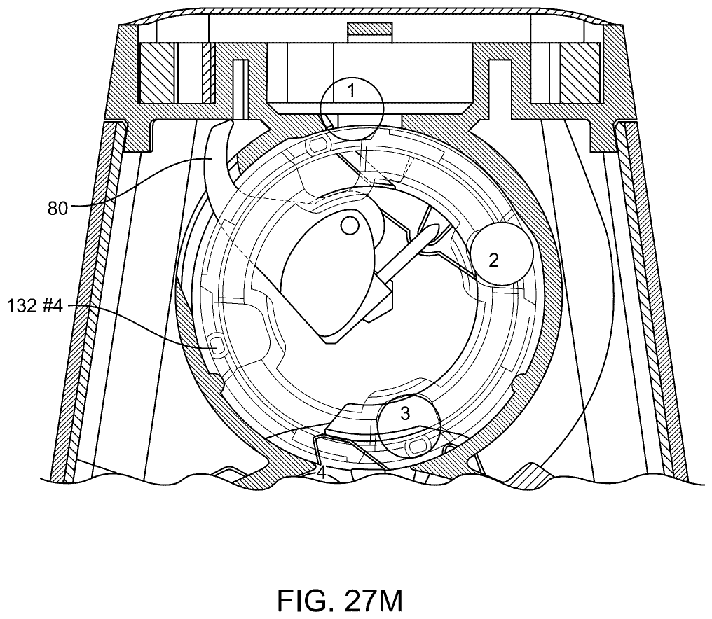

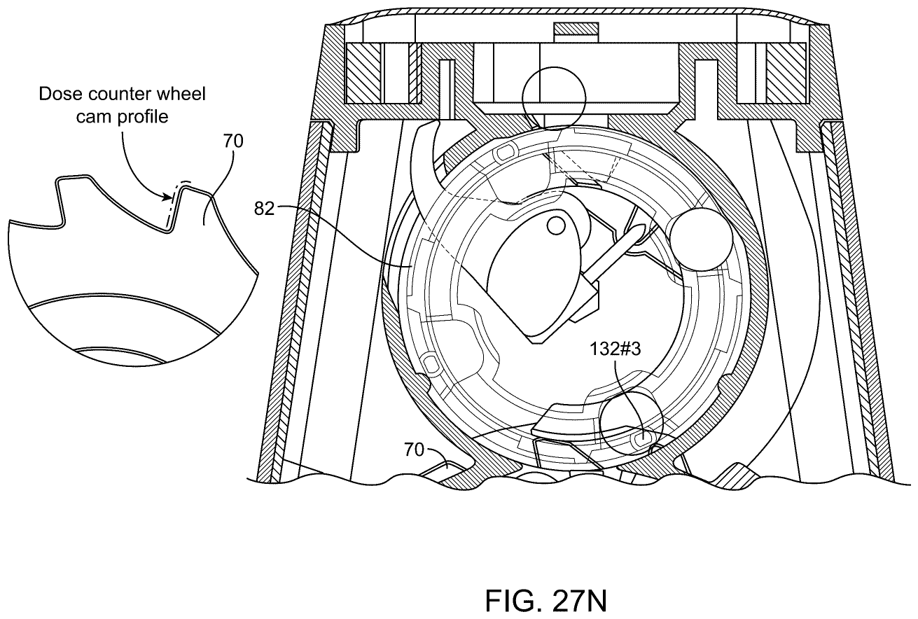

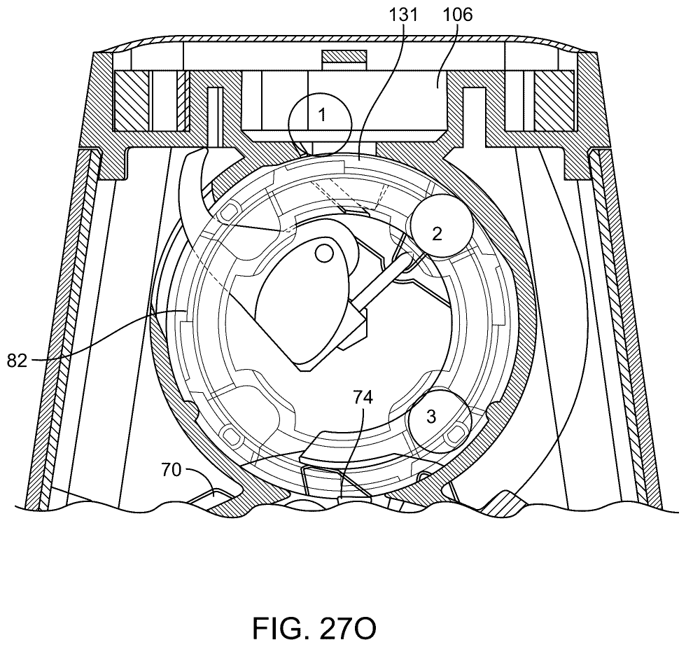

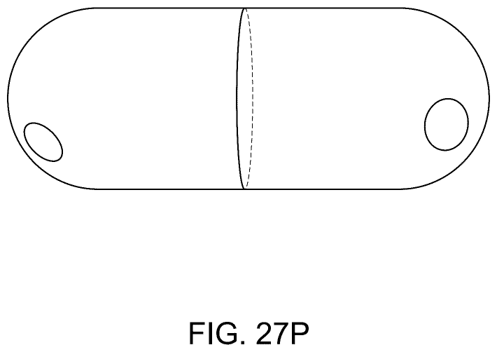

Downfall wheel 82 may be provided with solid surfaces 131 between the capsule spaces 128 for sealing off the bottom of the swirl chamber for use once a capsule has been loaded into the chamber so that air does not enter the chamber from the bottom, or airflow is at least reduced and any gap can be controlled to achieve a desired resistance as a system. As best seen in FIG. 24, the front edge of downfall wheel 82 may be provided with four forwardly protruding drive nubs 132 configured to engage with the outer teeth of dose count wheel 70 (shown in FIG. 27A) in a Geneva mechanism type manner such that each time downfall wheel indexes 90 degrees it drives dose count wheel 70 and attached carousel 74 through 22.5 degrees. In this exemplary embodiment, drive nubs 132 also serve to drive piercing hub 80, as will be subsequently described in detail. As best seen in FIG. 25, the rearward edge of downfall wheel 82 may be provided with four radially outward extending ratchet teeth 134 configured to cooperate with flexure arm 102 of drive plate 88 (shown in FIG. 16) to allow drive plate 88 to drive downfall wheel 82, as previously described.

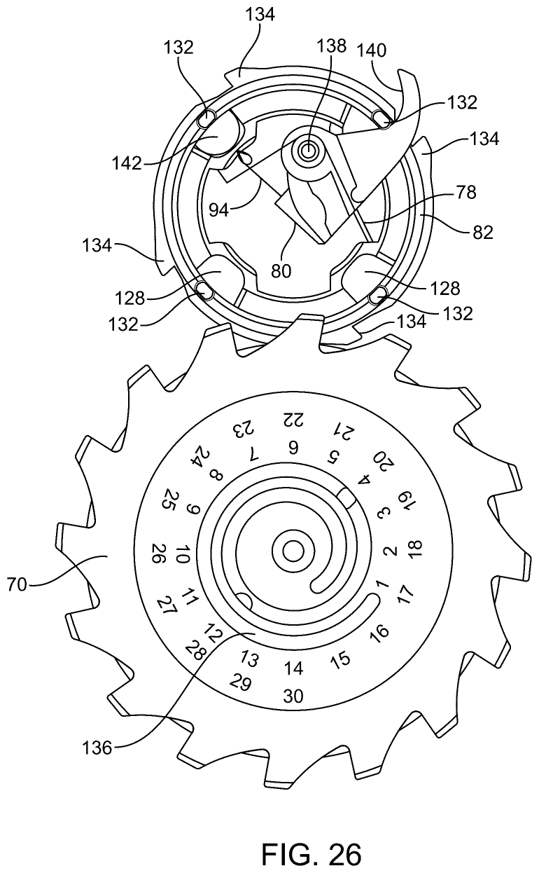

Referring to FIG. 26, details of dose count wheel 70 are shown. Dose count wheel 70 may be provided with a series of numbers with each number representing a number of capsules/doses remaining. In this exemplary embodiment, numbers 1 (or 0) through 30 are provided. In other embodiments (not shown), a larger or smaller number of doses may be represented. For example, 7, 14, 21, 28, 31, 35, 42, 45, 49, 56, 60, 61, 62, 63 or 70 doses may be represented (with the device configured with spaces for at least that many doses.) In some embodiments, some capsule spaces in a downfall wheel and or a carousel remain unused and do not have numbers associated with them on the dose count wheel 70 (other than 0.) In some embodiments, a downfall wheel may have only two capsule spaces spaced 180 degrees apart, with only one of the spaces preloaded with a capsule. In other embodiments, a downfall wheel may have 3, 5 or more capsule spaces evenly spaced around its circumference. In the current exemplary embodiment, the numbers 30 down to zero are arranged in a spiral extending from an outer radius to an inner radius of dose count wheel 70 and covering 675 degrees, as shown. In other embodiments (not shown), the spiral may extend from an inner radius to an outer radius, and or may extend a larger or smaller number of degrees.

A spirally extending groove 136 may be provided on the front side of dose count wheel 70 as shown. A mating protrusion (not shown) may be provided on the back side of sliding dose count window 68 (shown in FIG. 16.) With this arrangement, sliding window 68 is slowly driven vertically upward by dose wheel 70 as it turns counterclockwise (as viewed in FIG. 26) through 675 degrees. This ensures that only one number at a time shows through aperture 66 in front cover 52 (shown in FIG. 16.)

Referring to FIGS. 26 and 27A, details of piercing hub 80 are shown. FIG. 26 shows piercing hub 80 from the front while FIG. 27A shows it enlarged and from the back. As previously indicated, downfall wheel drive nubs 132 (shown in FIG. 26) serve to rotate hub 80 about hub rotation axis 138. Each time downfall wheel 82 rotates 90 degrees clockwise (as viewed in FIG. 26), one of the four drive nubs 132 engages with hub cam 140 to drive hub 80 through 77.29 degrees about its own axis offset from the downfall wheel rotation axis. The rotation ratio between the downfall wheel and hub 80 is not linear in this embodiment. As hub 80 rotates, a pair of piercing pins or sharps 94 (one forward and one rearward, so only one is seen in FIGS. 26 and 27A) are driven into capsule 142. Hub cam 140 may be shaped as shown to allow piercing pins 94 to track each capsule as it travels upward in downfall wheel 82 from position 1 (shown in FIG. 17) toward swirl chamber 106, with pins 94 continuing to generally point along a diameter of capsule 142 as the capsule moves. As hub 80 rotates, hub spring 78 is wound tighter. Capsule 142 is driven off the pins as it is pushed up the previously described ramps by the rotation of downfall wheel 82. This occurs before hub 80 returns to its start position. As downfall wheel 82 gets closer to the end of its 90-degree stroke, the tip of cam arm 140 clears drive nub 132, allowing hub spring 78 to rotate hub 80 back to its starting position as shown. Once hub 80 is back in its starting position, cam 140 is able to engage the next drive nub 132 and repeat the piercing process with the next capsule. This auto-piercing sequence is described in further detail below.

Referring to FIGS. 27B-27E, enlarged views of capsule piercing components and other interior components are shown. In particular, FIG. 27B shows piercing hub 80, downfall wheel 82 and associated components. FIG. 27C shows downfall wheel 82 and labels its four capsule recesses and four drive nubs. FIG. 27D shows piercing hub 80 and its cam features and profiles. FIG. 27E shows a portion of dose count wheel 70 and its cam features and profiles.

Referring to FIGS. 27F-27O, piercing hub 80 and downfall wheel 82 are shown in a sequence of states as they go through a piercing cycle. These views illustrate the capsule index mechanism sequence, the auto-pierce mechanism sequence, and the dose counter mechanism sequence of the present exemplary embodiment.

Starting with FIG. 27F, the auto pierce mechanism is shown in its initial position with capsule position 1, 2 and 3 identified. Clearance between the tips of the piercing pins 94 and the capsule in position 1 as shown ensures that the pins do not contact Capsule 2 before it moves into piercing position. In this initial position, drive nub #4 is just starting to contact the cam of piercing hub 80, and carousel 74 and dose count wheel 70 are in the “30” doses remaining position (not shown.)

In FIG. 27G, downfall wheel 82 has rotated counter-clockwise enough for drive nub 132 to rotate hub 80 counter-clockwise until piercing pin tips contact the hemi-spherical ends of Capsule 1.

FIG. 27H shows further advancement of downfall wheel 82 such that the piercing pins 94 have entered Capsule 1. Insertion depth will continue to increase as downfall wheel 82 and pin hub 80 rotate further. Drive nub #3 is now contacting one of the cam surfaces of dose wheel 70.

FIG. 27I shows the point in the piercing cycle where the pins reach their theoretical maximum insertion depth. Capsule #1 is pushed against the inside surface of chassis 76 to maximize pin insertion depth. Carousel 74 and dose count wheel 70 continue to rotate clockwise, driven by downfall wheel 82 nub #3. Capsule #3 begins to ramp up towards downfall wheel capsule recess #3.

In FIG. 27J, Capsule #1 contacts the bottom of the chassis ramp that will help lift it into swirl chamber 106. Dose count wheel 70 continues to rotate clockwise, driven by downfall wheel nub #3. Downfall wheel nub #4 continues to rotate hub 80 counterclockwise via the cam arm. Carousel 74 pushes Capsule #3 up the lower ramps towards downfall wheel capsule recess #3.

In FIG. 27K, downfall wheel nub #4 is sliding along the outer cam profile of hub 80 to hold the hub in a raised position as shown.

FIG. 27L shows downfall wheel 82 having rotated further counterclockwise from its position in FIG. 27K but hub 80 remains in the same position. In this position, downfall wheel 82 has rotated to the end of its hold rotation phase (i.e., nub #4 has reached the lower end of the outer cam profile of hub 80.) The additional rotation of downfall wheel 82 has driven Capsule #1 up the upper ramps, off of pins 94 and partway into swirl chamber 106.

FIG. 27M shows hub 80 after it has automatically returned to its initial rest position (driven by torsion spring 78, shown in FIG. 26) as downfall wheel nub #4 clears the hub cam profile.

FIG. 27N shows downfall wheel 82 further advanced, and dose count wheel 70 having been indexed to the next position, 22.5° clockwise from its starting position. Further counterclockwise rotation of downfall wheel 82 will cause drive nub #3 to skip past the upper end of the dose counter wheel cam profile.

In FIG. 27O, automatically pierced Capsule #1 has been delivered to swirl chamber 106. Downfall wheel 82 is now reset into the inhalation position with downfall wheel surface 131 shutting off the bottom of swirl chamber 106. Carousel 74 and dose counter wheel 70 are now in the “29” doses remaining position, and the capsule indexing, auto-piercing and dose counting sequences are set to start again.

FIG. 27P shows a representative medicine capsule after both hemispherical ends have been automatically pierced as described above.

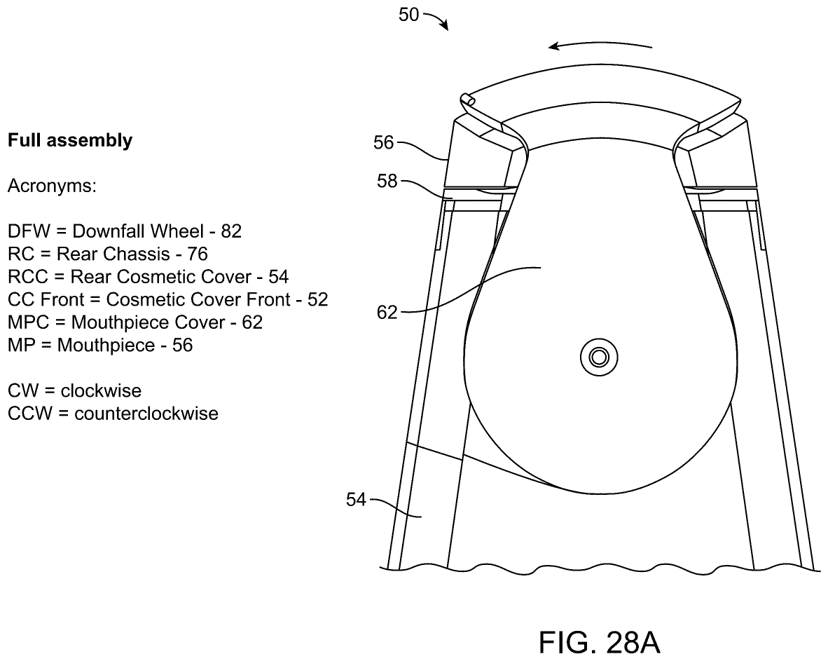

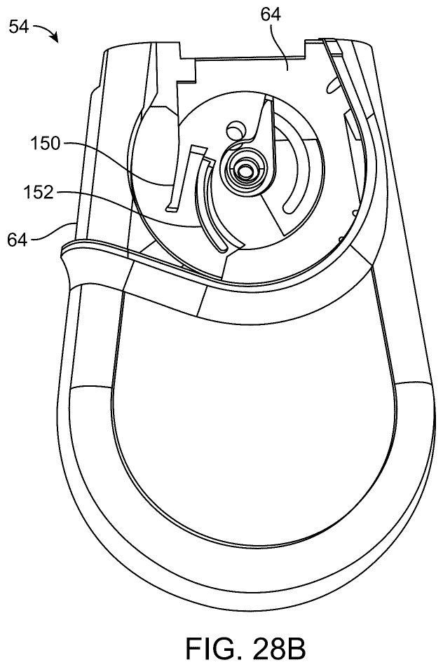

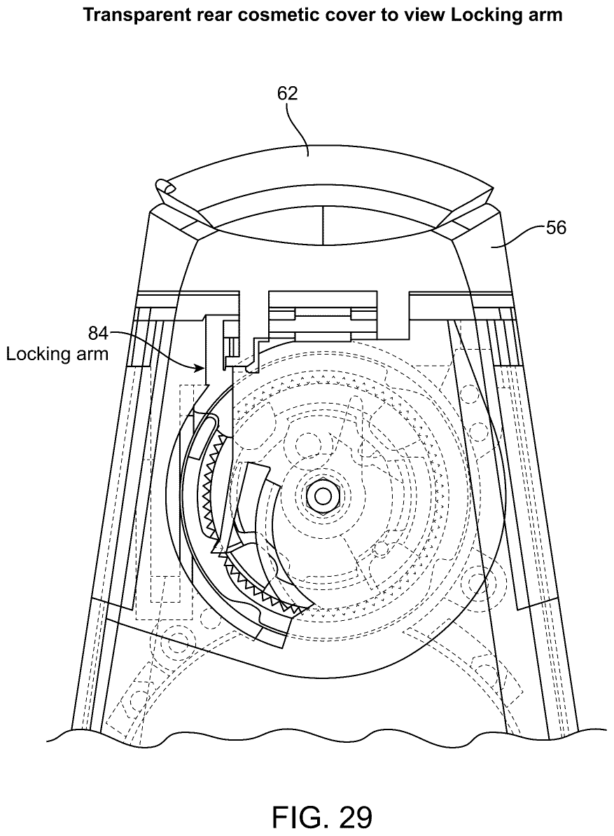

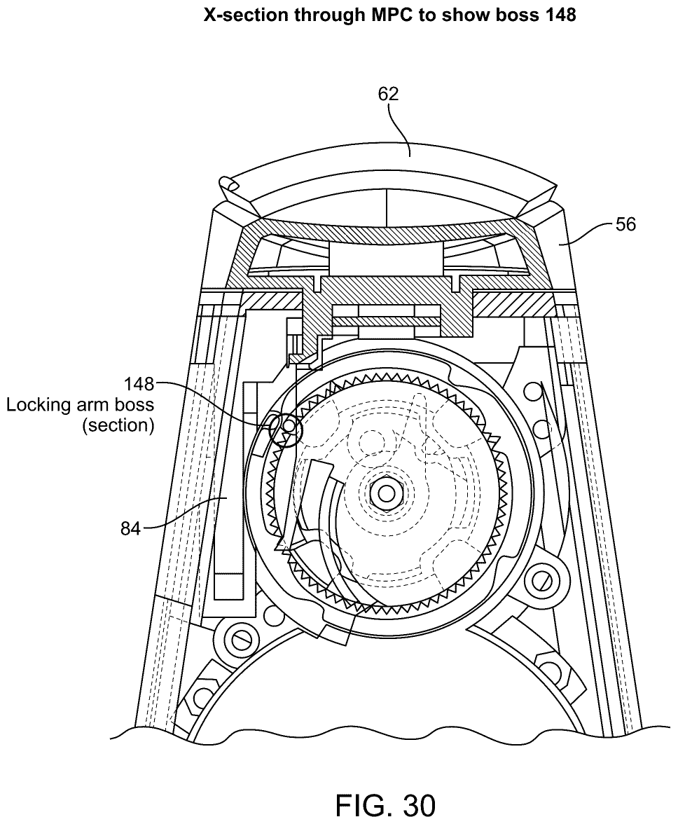

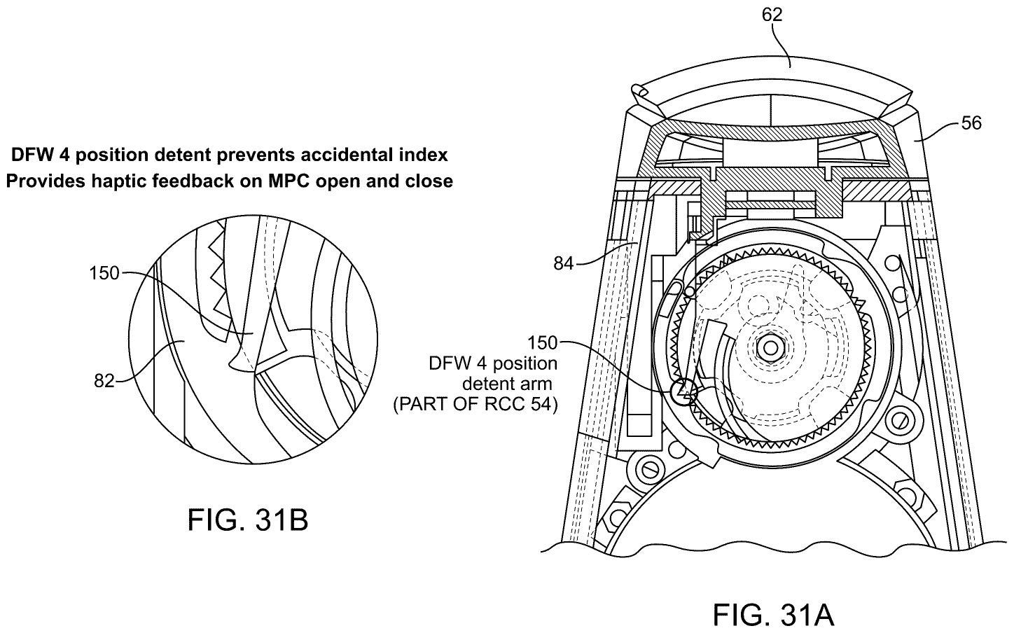

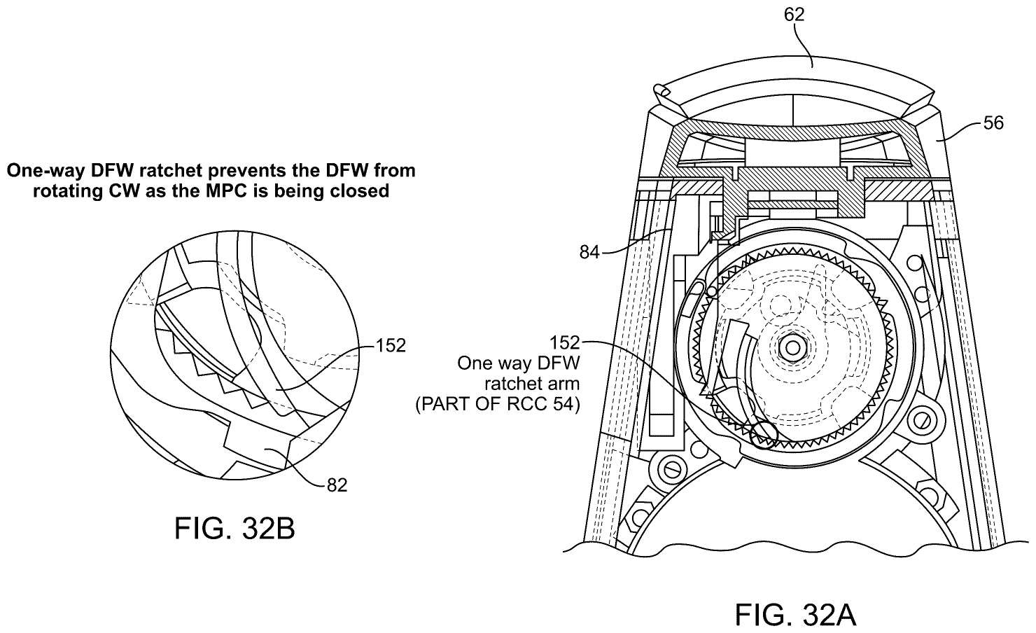

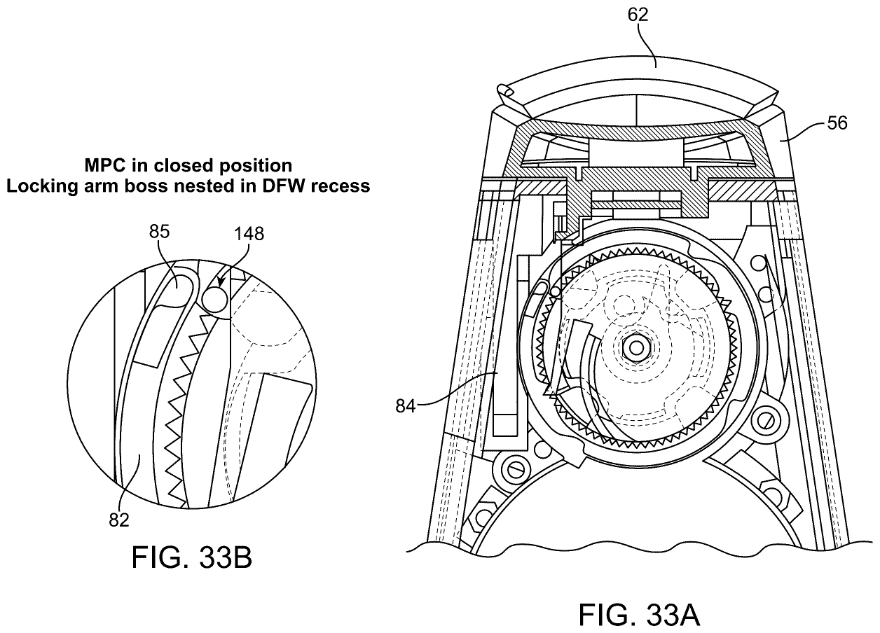

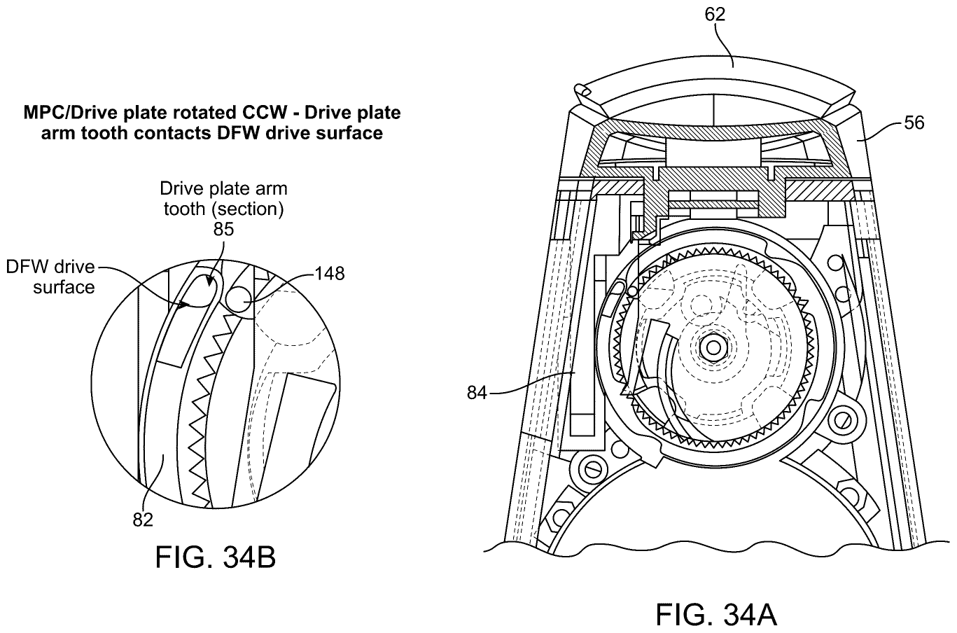

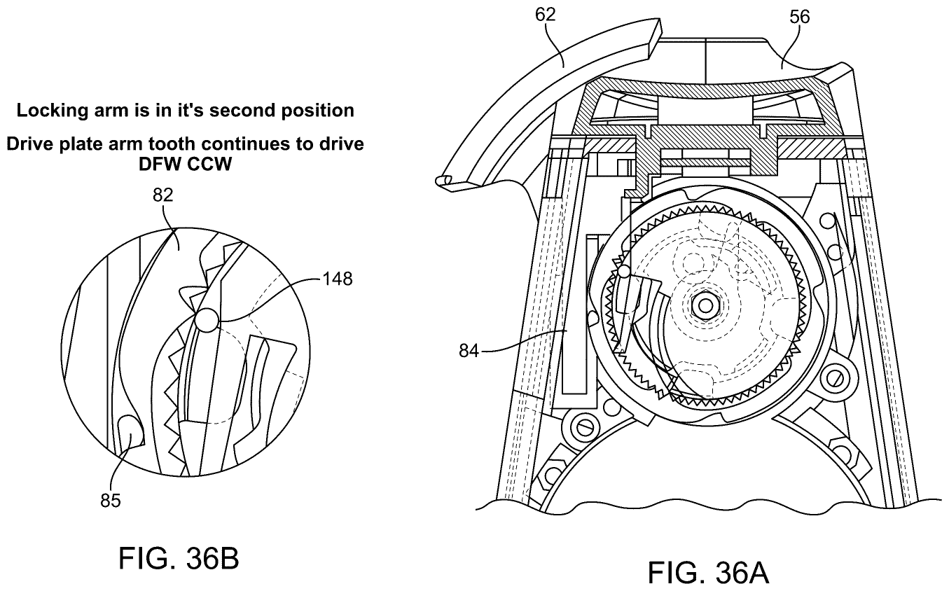

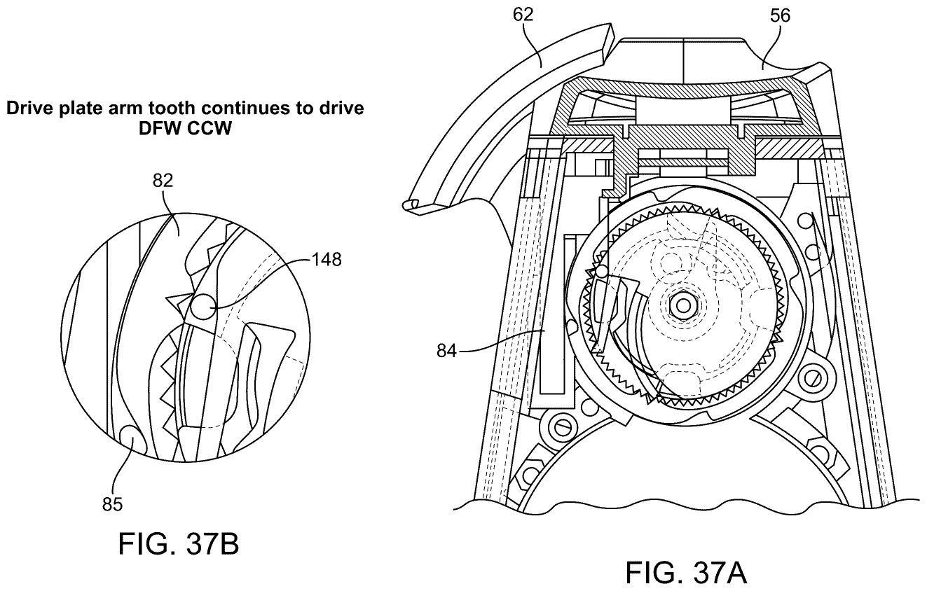

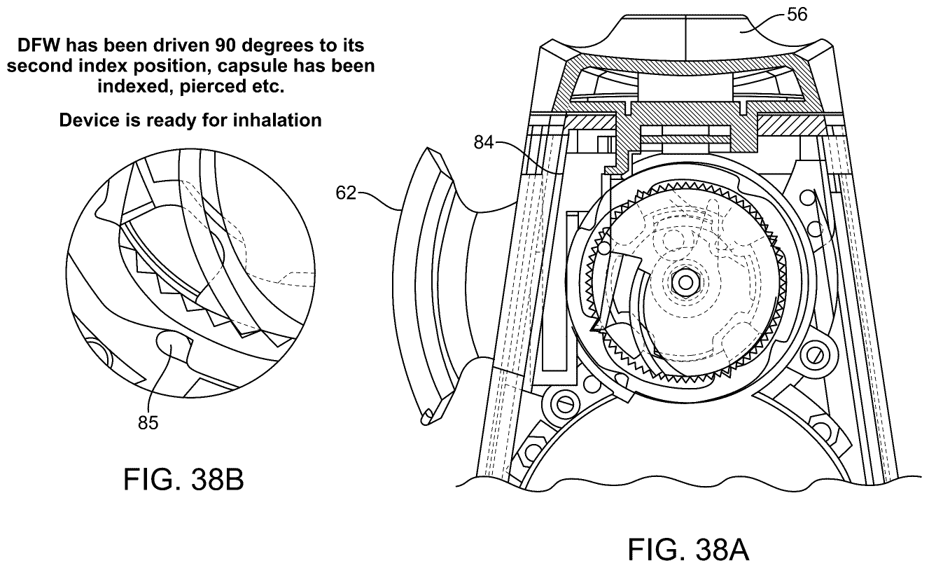

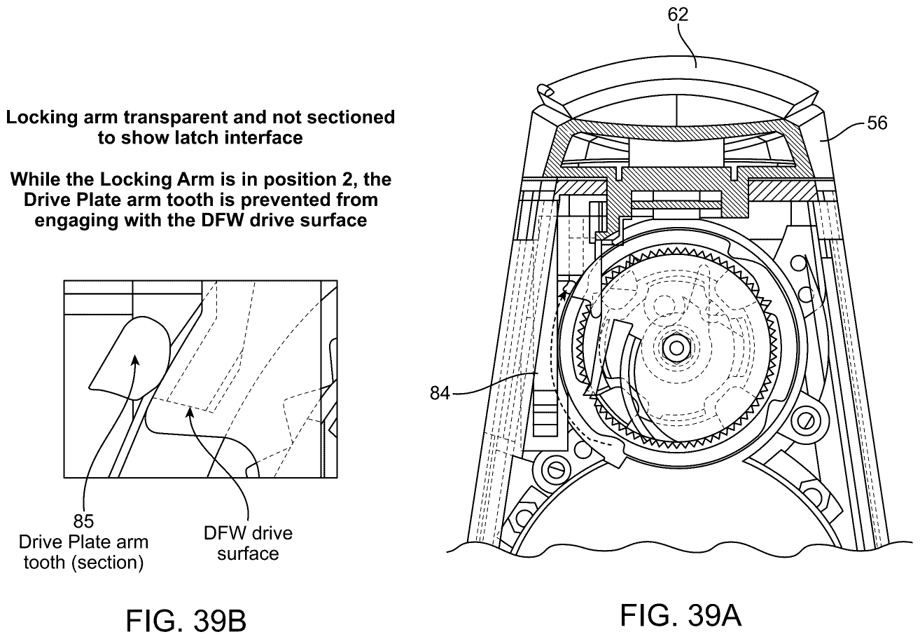

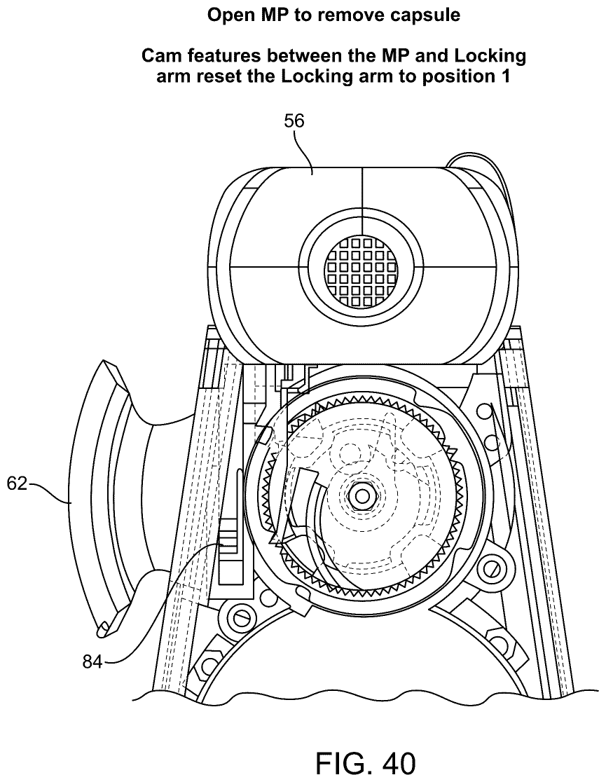

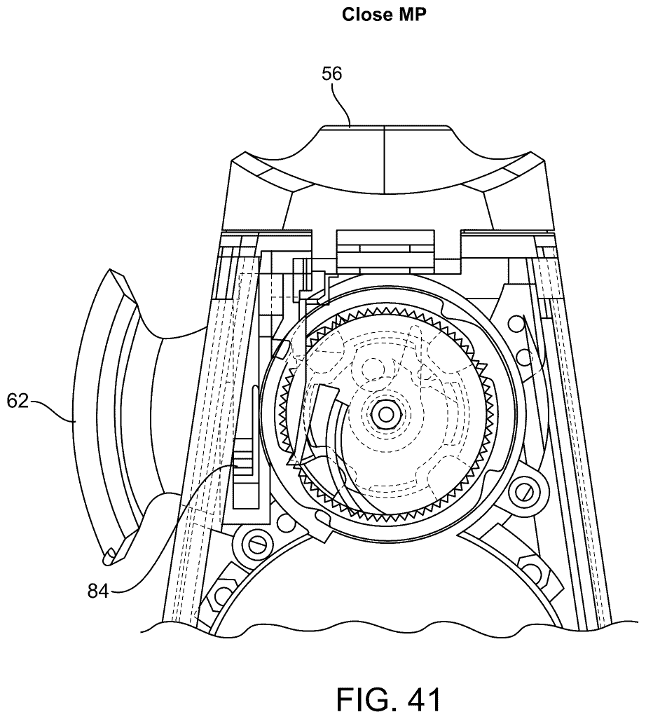

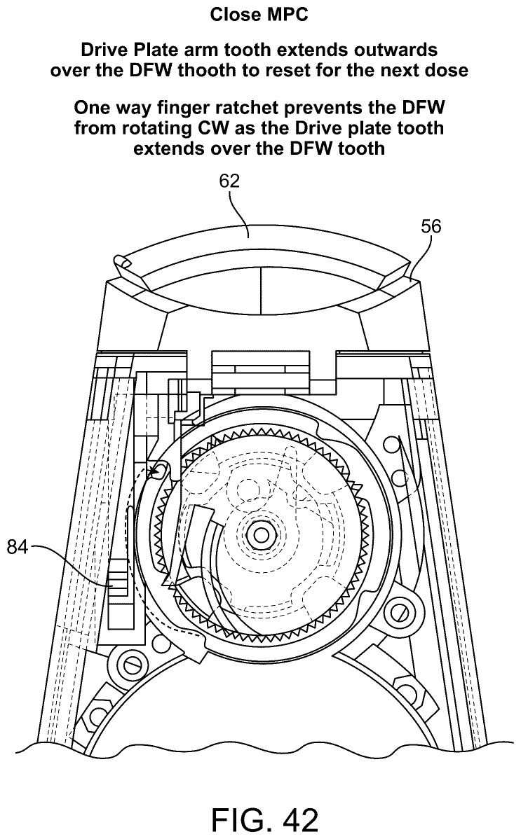

Referring to FIGS. 28A-46D, construction and operation of the mouthpiece cover interlock latch are shown. As previously referred to with reference to FIG. 15, in this exemplary embodiment, after a user inhales a dose from device 50, mouthpiece cover 62 can be moved to its closed position over mouthpiece 56 but will not remain there without springing back to the open position unless mouthpiece 56 has been opened and closed first. This signals to the user that the empty capsule that remains in the swirl chamber needs to be discarded before closing device 50 and putting it away, thereby readying the device for its next inhalation cycle.

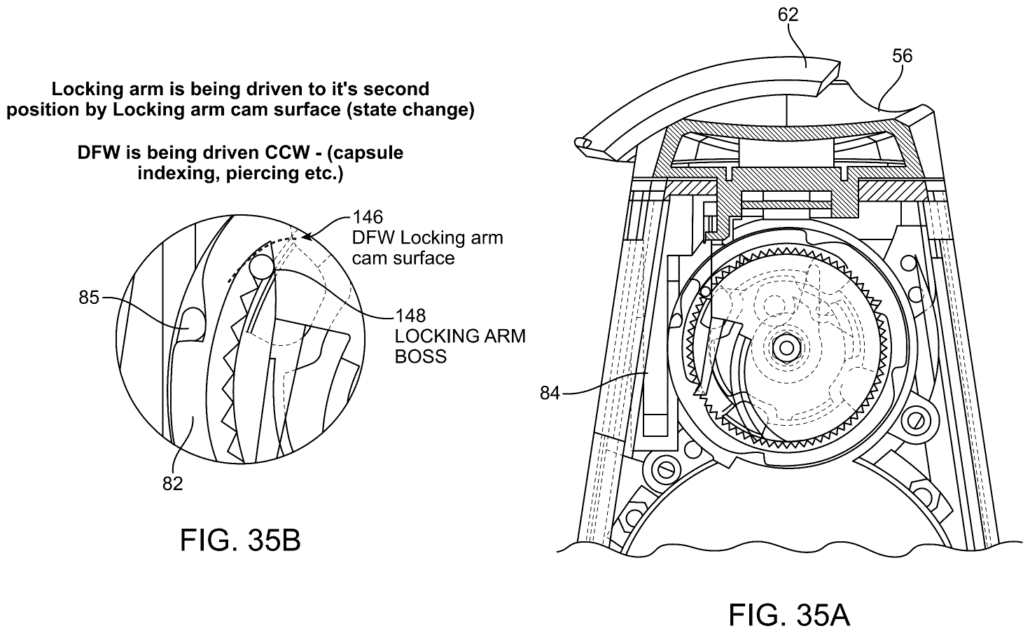

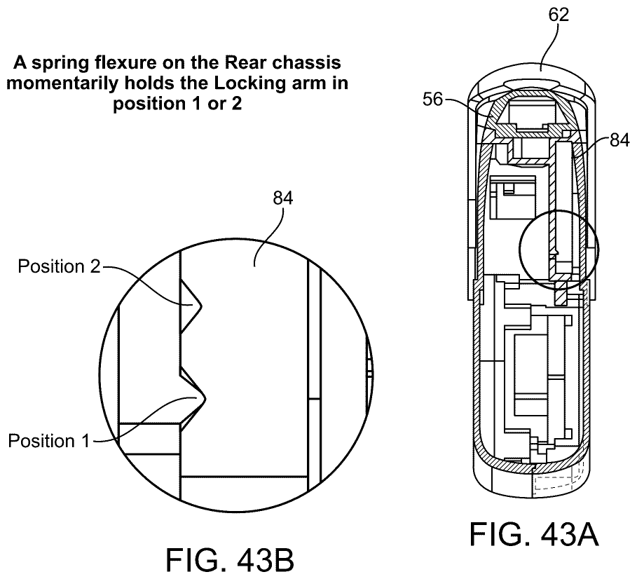

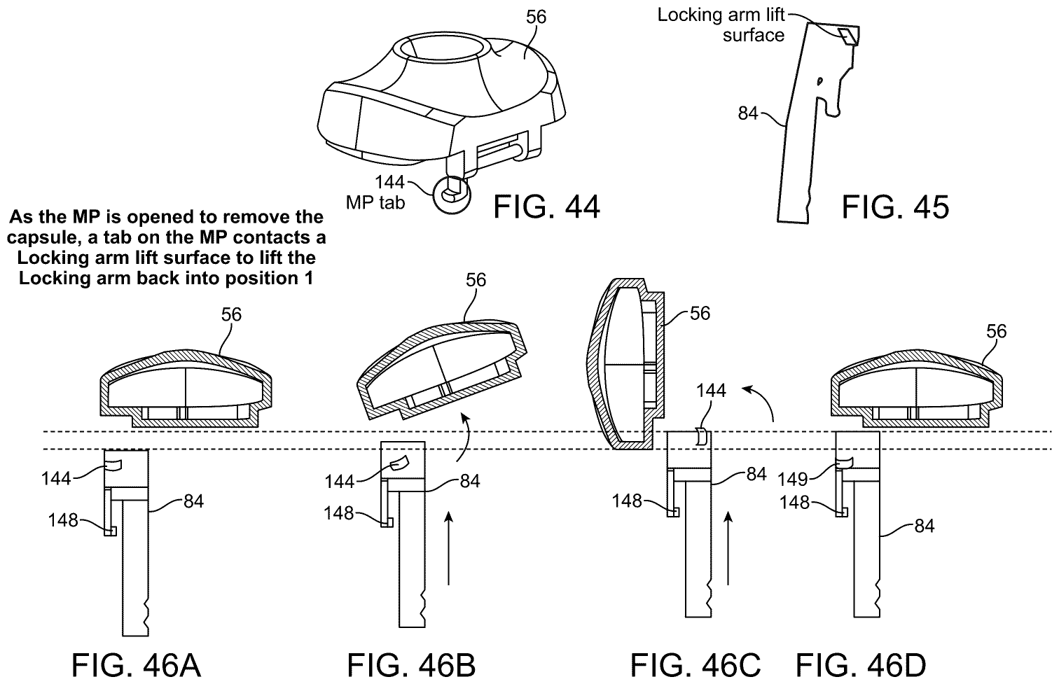

As best seen in FIGS. 43B and 46A-46D, latch arm 84 moves vertically between two states: an upper Position 1 in which the mouthpiece cover can be closed, thereby priming the device for its next cycle, and a lower Position 2 in which the mouthpiece cover will not remain closed and will not activate the next cycle. As shown in FIGS. 28A-46D, downfall wheel cam surface 146 (best seen in FIGS. 35A and 35B) is what drives latch arm 84 downward from Position 1 to Position 2 when mouthpiece cover 62 is being opened, and mouthpiece tab 144 (best seen in FIGS. 44 and 46A-46D) is what drives latch arm 84 upward from Position 2 to Position 1 when mouthpiece 56 is being opened. Further details are provided in FIGS. 28A-46D.

In some embodiments (not shown), inhaler device is configured without the above-described interlock features such that a mouthpiece cover may be opened and closed and another drug delivery cycle started without regard to whether the mouthpiece has been opened and closed first.

In the exemplary embodiment disclosed herein, inhaler device 50 is configured so that a user may hold the device and operate all of its functions with one hand, or may hold the device with one hand and operate it with the other hand. The cost of goods for device 50 is such that it can be recycled or otherwise disposed of after all its capsules have been depleted. In some implementations, device 50 may be reloaded with new capsules and reused after it has been cleaned and or sterilized. In some implementations, the inhaler device may be configured such that an empty carousel, cartridge or other capsule carrying device may be easily removed from the inhaler device by the end user or a provider of the inhaler device and replaced with a full capsule carrying device.

### Smart Device Components

In some embodiments, “smart device” features may be incorporated into inhaler device 50. For example, device 50 may be configured to record the time, date, location, doses remaining and or user input(s) whenever a dose cycle has been started and or completed. Some or all of this data and or additional data may be stored on the device for later retrieval and or it may be transmitted by wire or wirelessly to another device, such as a smartphone, tablet, laptop computer, desktop computer, computer network or other device. A data recording and or transmitting event may be triggered by a mouthpiece cover being opened or closed, by a mouthpiece being opened or closed, by an automatic sensing of airflow through a mouthpiece, by a user input, by a preset time and or by one or more other triggers. These events may be referred to as “delivery signatures” triggered by “a deflection component” activating a “sensor component”, such as a cam surface actuating a microswitch. Each of these components is now further described in greater detail.

### Sensor Component

As reviewed above, aspects of the disclosure include systems and devices that comprise a sensor component. Sensor components in accordance with embodiments of the disclosure are configured to acquire one or more data inputs from the subject systems and devices, or from the immediate vicinity of the subject systems and devices, and to transmit a report comprising a drug dose completion signal when a delivery signature is detected. In certain embodiments, the report transmitted by the sensor component includes additional information, such as, e.g., one or more drug identification characteristics (described further herein).

In some embodiments, a sensor component may include a sound sensor (e.g. a microphone) or pressure sensor to detect data related to the quality of the inhalation (e.g. peak flow rate, average flowrate, peak pressure, inhaled volume, duration of inhalation, etc.)

In some embodiments, a sensor component comprises a circuit board component that is configured or adapted to mechanically support and electrically connect one or more electronic components of a subject sensor. Circuit board components in accordance with embodiments of the disclosure can include, without limitation, printed circuit boards, etched circuit boards, flexible circuit boards, or any combination thereof. In some embodiments, a circuit board component comprises a printed circuit board (PCB).

Circuit board components in accordance with embodiments of the disclosure can comprise conductive tracks, pads, or other features that are etched from conductive sheets (e.g., copper sheets) that are attached to a non-conductive substrate. In certain embodiments, standard circuit components, such as, e.g., capacitors, resistors, memory components, and the like, are electrically connected to a circuit board component (e.g., are soldered to a PCB). Connection of one or more electronic circuit components to a PCB results in a printed circuit assembly (PCA) or a printed circuit board assembly (PCBA), which terms are used interchangeably herein.

Aspects of the disclosure include switches that are configured to establish or break an electrical contact in a subject circuit board component in response to an external stimulus (e.g., in response to an external mechanical stimulus). In some embodiments, a circuit board component comprises a momentary contact switch that is configured to establish or break an electrical contact only while the momentary contact switch is in an activated state. In some embodiments, a circuit board component comprises a non-momentary contact switch that is configured to establish or break an electrical contact until the non-momentary switch is activated again.

In some embodiments, a sensor component comprises a position sensor that is configured or adapted to permit position measurement of one or more components of the subject drug delivery systems and devices. For example, in some embodiments, a position sensor is configured to detect and/or measure a position of an actuation component and/or a deflection component. In some embodiments, a position sensor is configured to detect an orientation of one or more components of a subject device. Position sensors in accordance with embodiments of the disclosure can be absolute position sensors or relative position sensors, and can be linear, angular or multi-axis position sensors. In some embodiments, a position sensor is configured to acquire a plurality of measurements over a defined time interval, or during execution of a drug delivery procedure, in order to measure a position of one or more components of the subject systems or devices, either as a function of time, or as a function of progression through the drug delivery procedure.

In some embodiments, a sensor component and/or a deflection component comprises a force sensor that is configured or adapted to detect and/or measure one or more forces in one or more components of the subject drug delivery systems and devices. Force sensors in accordance with embodiments of the disclosure can be absolute or relative force sensors. Non-limiting examples of force sensors include electrical resistance strain gauges, elastic strain gauges, foil strain gauges, semiconductor strain gauges, thin-film strain gauges, wire strain gauges, piezoelectric force transducers, strain gauge load cells, inductive sensors, and the like.

In some embodiments, a sensor component comprises a light sensor that is configured or adapted to detect and/or measure ambient light. For example, in some embodiments, a light sensor is configured to determine whether an amount of ambient light in the vicinity of a subject drug delivery system or device is above a predetermined threshold value. Light sensors in accordance with embodiments of the disclosure can be absolute or relative light sensors. In some embodiments, a light sensor is used to detect an increase in ambient light, thereby indicating that a subject device has been removed from its packaging, removed from a storage container, and/or removed from a dark location.

In some embodiments, a sensor component comprises a motion sensor that is configured or adapted to detect and/or measure motion of a subject drug delivery system or device. For example, in some embodiments, a motion sensor is configured to determine whether a device, or component thereof, moves more than a predetermined threshold value. Motion sensors in accordance with embodiments of the disclosure can be absolute or relative motion sensors. In some embodiments, a motion sensor is used to detect motion of a subject device, thereby indicating that a user has begun interacting with the device.

In some embodiments, a sensor component comprises a temperature sensor that is configured or adapted to detect and/or measure a temperature of one or more components of the subject systems or devices. For example, in some embodiments, a temperature sensor is configured to determine whether the temperature of a drug is above a predetermined threshold value or is within a predetermined temperature range. Temperature sensors in accordance with embodiments of the disclosure can be absolute or relative temperature sensors. In some embodiments, a temperature sensor is used to detect an increase in temperature, thereby indicating that a subject device has been removed from cold storage and has reached a temperature that is suitable for administration of the drug to a patient. In some embodiments, a temperature sensor is used to determine when a cold chain is broken (i.e., when the temperature of the device or a portion thereof rises above a predetermined threshold temperature) and to record this information. In some embodiments, a temperature sensor is used to track when the device or a portion thereof rises above a predetermined threshold temperature, and to wake up the device when the temperature reaches the predetermined threshold temperature to record an inhalation procedure. Any information relating to the cold chain of the device can be recorded and used for purposes of information tracking and/or for preparing the device for use. In some embodiments, the device is configured to wake up from a deep sleep, read the temperature from the sensor and go back to sleep. This process may be performed with low power levels once per minute or other frequency to allow long term storage of the device. In some embodiments, this process may be performed for up to 5 years on a single battery, due to fast action of the microprocessor and selective power up of only circuit components needed to read a temperature.

In some embodiments, a sensor component comprises a touch sensor that is configured or adapted to detect and/or measure contact by an object that is conductive, or that has a dielectric value that is different from air. In some embodiments, a touch sensor comprises one or more detection components (e.g., capacitive sensing components) that are placed in close proximity to, or on, the inside of an external surface of a subject drug delivery system or device and are electrically connected to the touch sensor. When a user touches a detection component, an electrical signal is sent to the touch sensor, indicating that the user has touched the device. In some embodiments, a touch sensor is used to determine that a user has made physical contact with a subject device (e.g., that a portion of a user's skin has made physical contact with a subject device), thereby indicating that the user has begun interacting with the device.

Aspects of the subject sensor components include a power component that is configured or adapted to provide electrical power to the sensor component. In some embodiments, a power component comprises a battery. In some embodiments, a power component comprises a rechargeable battery. In certain embodiments, such as, e.g., where one or more components are disposable, a power component does not include a rechargeable battery. In some embodiments, a power component comprises one or more standard electrical cords that are configured to supply electrical power to a sensor component by establishing electrical contact with an external power source (e.g., a standard electrical outlet). In some embodiments, a subject system or device comprises an on/off switch or button that can be used to turn power to the system or device on or off, as desired.

In some embodiments, a sensor component comprises a memory component that is configured or adapted to store one or more drug identification characteristics therein. Memory components in accordance with embodiments of the disclosure can be volatile or non-volatile memory components. In some embodiments, a memory component is encoded with one or more drug identification characteristics before it is connected to the sensor component (e.g., the memory component is encoded with one or more drug identification characteristics at the time the memory component is manufactured). In some embodiments, a memory component is encoded with one or more drug identification characteristics after the memory component has been connected to the sensor component. In certain embodiments, a sensor component comprises a data acquisition component that is configured to acquire the one or more drug identification characteristics that are stored in the memory component from an external source (e.g., from an external encoder, or from a memory component on a drug carousel or cartridge). In some embodiments, a memory component is configured to wirelessly receive encoded information (e.g., a data acquisition component is configured to wirelessly acquire the one or more drug identification characteristics). In some embodiments, a sensor component comprises a near-field communication (NFC) component and/or a radio frequency identification (RFID) component that are configured for data exchange.

Drug identification characteristics in accordance with embodiments of the disclosure broadly include any information relating to a drug's identity and/or its biochemical characteristics (including, but not limited to, a drug's name, concentration, dose, dosage, serial number, lot number, universal unique identifier (UUID), expiration date, manufacturing date, site of manufacture, or any combination thereof). In some embodiments, a memory component can further comprise one or more patient identification characteristics (including, but not limited to: a patient name, patient identification number, prescription number, demographic information, patient group or subgroup, or any combination thereof). In some embodiments, a memory component can further comprise one or more drug delivery device identification characteristics (including, but not limited to: a system or device name, type, model number, serial number, lot number, date of manufacture, place of manufacture, UUID, or any combination thereof). In some embodiments, a memory component is configured to be programmed (e.g., during manufacture of the device) using over-the-air transmission with a universal unique identifier (UUID).

Aspects of the subject sensor components include a wireless transmitter module that is configured to wirelessly transmit data to a networked device (e.g., a data management component). In some embodiments, a networked device is a secure networked device. In some embodiments, transmitted data can be encrypted. In some embodiments, a wireless transmitter module is configured to communicate with one or more networked devices using a wireless transmission component (e.g., a communication link that utilizes, e.g., infrared light, radiofrequency, optical or ultrasound waves, or any combination thereof). Networked devices in accordance with embodiments of the disclosure broadly include any device or component that communicates with at least one other device over a communication link. Non-limiting examples of networked devices include mobile computing devices (e.g., smart phones, laptop computers) that use, e.g., Bluetooth, Bluetooth low energy (BLE), or Wi-Fi connections. In some embodiments, a wireless transmitter module is configured to wirelessly communicate directly with a network or directly with a remote computing device (i.e., without first communicating with a mobile computing device). In certain embodiments, a wireless transmitter module comprises an antenna. Aspects of the disclosure broadly include any radio wave spectrum communication systems, including but not limited to those that can communicate to a central hub, and then into a cloud-based computing/data transmission environment.

Sensor components in accordance with embodiments of the disclosure are configured to transmit a report comprising a drug dose completion signal when the sensor component detects a delivery signature. In some embodiments, a drug dose completion signal comprises an indication that an actuation component has completed a delivery stroke. In some embodiments, a data management component is configured to determine a volume of drug that was delivered to the patient by identifying the drug delivery system or device and determining the volume of drug that is administered in a single delivery stroke of the identified system or device. In some embodiments, a data management component is encoded with information relating to, e.g., a volume of a drug that is administered in a single delivery cycle of a specified system or device.

In some embodiments, a subject sensor component is configured or adapted to determine one or more operational states of a drug delivery system or device. For example, in some embodiments, a sensor component is configured to determine a ready state, wherein the system or device is ready to administer a drug dose to the patient. In some embodiments, a sensor component is configured to determine an unready state, wherein the system or device is not ready to administer a drug dose to the patient. In some embodiments, a sensor component is configured to determine a dose-in-progress state, wherein the system or device is actively administering a drug dose to the patient. In some embodiments, a subject system or device can be configured to administer a drug dose to a patient over a time frame that ranges from about 1 second up to about 30 minutes, such as about 5, 10, 15, 20, 25, 30, 35, 40, 45, 50, 55, or 60 seconds, or about 1, 2, 3, 4, 5, 6, 7, 8, 9 or 10 minutes or more, such as about 11, 12, 13, 14, 15, 16, 17, 18, 19, 20, 21, 22, 23, 24, 25, 26, 27, 28 or 29 minutes or more. In some embodiments, a subject system or device is configured to remain in a dose-in-progress operational state for a period of time that is equal to the time frame for administering the drug to the patient.

In some embodiments, a sensor component is configured to determine a sleep mode state (e.g., a low power state), wherein the system or device is operating in reduced power mode and is not ready to administer a drug dose to the patient. In some embodiments, a sensor component is configured to determine a low battery state, wherein the battery charge is below a predetermined level.

Determination of any of the states described herein can be accomplished by analysis of one or more inputs from one or more of the subject sensor components. For example, in some embodiments, a ready state can be determined when a temperature value from a temperature sensor falls within a predetermined range (i.e., indicating that the drug is at a desired temperature range for administration) and a position sensor indicates that the system or device is in a desired position or orientation for administration (e.g., a position of an actuation component is determined to be correct for administration of the drug to the patient). This arrangement can also be used for training the user on correct ‘inhalation posture’ orientation. In some embodiments, this may be done in conjunction with the correct inhalation posture orientation being shown on the graphical user interface of the user's mobile device.

In some embodiments, a sensor component can communicate a determined operational state, as described above, to another component of the system or device (e.g., to a data management component). In certain embodiments, the data management component can then indicate the operational state to a user (e.g., on a GUI), thereby communicating the operational state to the user. In some embodiments, as described further herein, the subject systems and devices can comprise one or more indicator components that are configured to communicate an operational state of the system or device to a user (e.g., a “ready to inhale” operational state).

Sensor components in accordance with embodiments of the disclosure can be mounted in any suitable location on the subject systems or devices. For example, in some embodiments, a sensor component can be mounted in a housing that is positioned anywhere on the system or device. In certain embodiments, a sensor component is formed into a single unit. In certain embodiments, a sensor component comprises two or more individual units (e.g., two or more different PCBAs) that are electrically connected to one another, each of which is mounted in a suitable location on a subject drug delivery system or device.

### Deflection Components

Aspects of the disclosure include one or more deflection components that are configured to generate a delivery signature when a delivery stroke has been completed. The subject drug delivery systems and devices are configured to transmit a report comprising a drug dose completion signal only when a delivery signature has been generated and detected.

Deflection components in accordance with embodiments of the disclosure can be positioned in any suitable location on the subject systems or devices so that they can interact with one or more components of the subject systems and devices during the execution of a delivery cycle. In some embodiments, a deflection component is located along the length of an actuation component and is configured to be mechanically deflected by at least a portion of the system or device during a delivery cycle. In some embodiments, a deflection component comprises a force sensor that is configured to measure one or more forces applied to a portion of a subject system or device by a user. In some embodiments, a deflection component comprises one or more inductive sensor coils that are configured to move toward one or more detection targets in response to a force applied to one or more components of a subject system or device.

As reviewed above, in some embodiments, a deflection component comprises a force sensor. Force sensors in accordance with embodiments of the disclosure can be absolute or relative force sensors, and details of such sensors are generally known in the art.

In certain embodiments, a deflection component comprises one or more inductive sensor coils that are configured to move toward a detection target in response to a force that is applied to a portion of the subject systems and devices during a delivery stroke. Inductive sensor coils in accordance with embodiments of the disclosure generally operate by generating an alternating electrical field that can detect a conductive material within a certain proximity of the inductive sensor coil. As such, inductive sensor coils in accordance with embodiments of the disclosure generally operate in conjunction with one or more detection targets that comprise a conductive material (e.g., a conductive metal material). The configuration of an inductive sensor coil and its detection target can take on any suitable arrangement. For example, in some embodiments, a first inductive sensor coil is disposed on a base portion of a deflection component, and the base portion is configured to deflect towards the detection target when a force is applied to the base portion by a user during a delivery stroke. In some embodiments, an inductive sensor coil is disposed on an extension component that is configured to move through a portion of a subject device during a delivery stroke and is configured to pass one or more portions of a detection target during the delivery stroke.

Detection targets in accordance with embodiments of the disclosure can have any number of different geometries. For example, in some embodiments, a detection target has a uniform geometry that does not change significantly as function of position in a given direction. Non-limiting examples of uniform geometry detection targets include geometric shapes (e.g., rings, bands, rectangles, and squares). In some embodiments, a uniform geometry detection target can be placed on an internal or an external surface of a subject device and can be detected when an inductive sensor coil passes the detection target and/or moves towards it. In certain embodiments, a detection target with a uniform geometry is disposed over at least half of a surface of a subject system or device.

In some embodiments, a detection target has a repeating geometry, wherein two or more uniform geometry detection targets are disposed in a repeating manner in a given direction along a surface of a subject device. For example, in some embodiments, two or more circular or square detection targets can be disposed in series along the length of a device component. As an inductive sensor coil passes each uniform target, the progression of the delivery stroke can be determined.

In some embodiments, a detection target has a variable geometry, wherein one or more dimensions of the detection target change as a function of position in a given direction. For example, in some embodiments, a variable detection target comprises a conductive material that starts at a minimum width at a first end of a device component, and the width gradually increases along the length of the component to a final maximum width at the other end of the component. Any number of variations in the variable geometry can be introduced to generate a unique reading, or signature, that is obtained as an inductive sensor coil moves past the variable geometry detection target.

### Delivery Signatures

As reviewed above, aspects of the disclosure include detection of a delivery signature by a subject sensor and/or data management component. Delivery signatures in accordance with embodiments can comprise any of a variety of data components. For example, in embodiments where a deflection component comprises a plurality of trigger switches, a delivery signature can comprise data relating to a deflection order of the trigger switches, a deflection duration of each trigger switch, one or more time intervals corresponding to a time between a deflection of a first trigger switch and a deflection of a second trigger switch, or any combination thereof.

In embodiments where a deflection component comprises a first and second inductive sensor coil and a first and second detection target, a delivery signature can comprise a detection signal from the first and second inductive sensor coils, corresponding to detection of the first and second detection targets. In such embodiments, where the second detection target comprises a uniform geometry, a delivery signature can comprise detection of the uniform geometry of the second detection target by the second inductive sensor coil, which can be located on an extension component of the deflection component. Further, in such embodiments, where the second detection target comprises a repeating geometry, a delivery signature can comprise detection of the repeating geometry by the second inductive sensor coil, which can be located on an extension component of the deflection component. Additionally, in embodiments where a detection target comprises a variable geometry, a delivery signature can comprise a detection signal from one or more attributes of the variable geometry, such as a proportional signal.

Delivery signatures in accordance with embodiments of the disclosure can have a characteristic shape that is indicative of an inhalation profile. Aspects of the disclosure involve detecting all or a portion of a delivery signature in order to measure a progression of a delivery profile or a completion of a delivery profile. In some embodiments, a delivery signature is detected and/or analyzed by a sensor component. In some embodiments, a delivery signature is detected and/or analyzed by a data management component. In some embodiments, a delivery signature is compared to a reference signature, and if the delivery signature matches the reference signature within acceptable tolerance levels, a successful delivery profile is recorded. In some embodiments, a data management component is programmed with one or more algorithms that are adapted to apply a set of rules to determine whether a delivery signature sufficiently conforms to a predetermined reference signature.

In one embodiment, a delivery signature is generated when a plurality of trigger switches is deflected in a given direction for a specified period of time. For example, when all of the trigger switches in a trigger switch assembly are deflected in for a specified period of time (e.g., for 3 or more seconds, such as 4, 5, 6, 7, 8, 9, and 10 or more seconds), the data management component determines that the delivery signature conforms to a reference signature.

### Actuation Component

Aspects of the disclosure include actuation components that are configured to move, thereby causing a capsule to be dispensed from a capsule space into a swirl chamber. Actuation components in accordance with embodiments of the disclosure can generally be actuated by any suitable mechanism. In some embodiments, an actuation component is configured to be moved manually by a user. In some embodiments, an actuation component is configured to be moved automatically by one or more driver components (e.g., one or more mechanical, electrical, or electromechanical controllers).

In some embodiments, an actuation component can include a controller that is coupled to one or more assemblies or subassemblies of the subject systems or devices. The controller can be configured or adapted (e.g., programmed, if the controller comprises an electrical or electromechanical component) to move the actuation component in response to a user input or an activation signal.

In some embodiments, an actuation component can comprise one or more coupling components that are configured or adapted to mechanically connect the actuation component to one or more additional components of the subject systems or devices. Coupling components in accordance with embodiments of the disclosure broadly include threaded couplers, adhesive couplers, snap-fit couplers, magnetic couplers, or any combination thereof.

### Indicator Component

Aspects of the disclosure include indicator components that are configured or adapted to communicate one or more operational states of the subject drug delivery systems or devices to a user. In use, a given operational state of a subject drug delivery system or device can be assigned a specific indicator signal, and the subject indicator components can be used to communicate the specific indicator signal to a user, thereby indicating to the user that the system or device is in the indicated operational state. Indicator components in accordance with embodiments of the disclosure broadly include visual, haptic and auditory indicators, each of which is described in further detail herein.

Aspects of the disclosure include visual indicator components that are configured or adapted to display a visual signal regarding an operational state of a subject system or device to a user. In some embodiments, a visual indicator comprises a light-emitting component. Light emitting components in accordance with embodiments of the disclosure include, without limitation, light emitting diodes (LEDs) and organic light emitting diodes (OLEDs). In some embodiments, a visual indicator comprises a light pipe (also referred to as a light tube). In some embodiments, a light pipe comprises a hollow structure that is configured to contain light within the structure by utilizing a reflective lining. In some embodiments, a light pipe comprises a transparent solid material that is configured to contain light within the material by utilizing total internal reflection. In some embodiments, a visual indicator comprises a diffuser component (e.g., a light pipe diffuser) that is configured to uniformly spread a visual signal (e.g., light from an LED) over a defined area. In some embodiments, a visual indicator component comprises a light pipe and a light pipe diffuser. Visual indicator components in accordance with embodiments of the disclosure can be configured or adapted to generate visual signals having any color (e.g., red, orange, yellow, green, blue, purple) or any combination thereof. In some embodiments, an operational state of a subject system or device can be assigned a specific color. For example, in one embodiment, an unready operational state is assigned the color red, and when the system or device is in an unready state, a red color is displayed to a user using a visual indicator component. In some embodiments, a visual indicator component can be configured to flash a visual indicator on and off in a particular sequence (e.g., a series of three short flashes) or to remain constantly on to provide an indication of an operational state. Other indicator settings in accordance with embodiments of the disclosure include attempting to connect to a data management component (e.g., flashing yellow or blue indicator light), and connected to a data management component (e.g., flashing or solid green or blue indicator light). Any of a variety of “ready” or “unready” states can be indicated to a user. For example, in some embodiments, an indicator component is configured to indicate that a capsule storage component has reached a suitable temperature for use. In some embodiments, an indicator component is configured to indicate to a user that the device is attempting to connect to a wireless network or a data management component. In some embodiments, an indicator component is configured to indicate to a user that the device is connected to a wireless network or a data management component. In some embodiments, indicator component(s) can be located on a GUI of a mobile device.

Aspects of the disclosure include haptic indicator components that are configured or adapted to generate one or more vibration signals that are specific to an operational state of a subject system or device. In some embodiments, a haptic indicator comprises a vibration generator component. Vibration generator components in accordance with embodiments of the disclosure are configured or adapted to generate vibrations having any desired combination of amplitude, frequency and duration in order to generate a plurality of unique vibration signals. For example, in one embodiment, an unready operational state can be assigned a vibration signal that consists of a single, high amplitude vibration that has a duration of one second.

Aspects of the disclosure include auditory indicator components that are configured or adapted to generate one or more auditory signals that are specific to an operational state of a subject system or device. In some embodiments, an auditory indicator comprises a sound generator component. Sound generator components in accordance with embodiments of the disclosure are configured or adapted to generate a plurality of unique sounds having a plurality of different tones and/or volumes. For example, in one embodiment, an unready operational state can be assigned a sound that consists of a single, high-volume buzzer sound.

Indicator components in accordance with embodiments of the disclosure can be mounted in any suitable location on the subject systems or devices. For example, in some embodiments, an indicator component can be mounted in a housing that is positioned anywhere on the system or device. In some embodiments, an indicator component can comprise a plurality of individual components that work in concert to generate a desired indicator signal. For example, in one embodiment, a visual indicator component comprises an LED that generates a visible light signal, and also comprises a light pipe that transfers the visible light from the LED to one or more locations on the subject system or device. In some embodiments, a visual indicator further comprises a light pipe diffuser that evenly spreads the visible light signal over a desired location (e.g., over an entire indicator window).

### Housing Component

Aspects of the disclosure include one or more housing components that are formed from suitable materials, such as, e.g., ceramic, plastic, metal, or any combination thereof. In some embodiments, one or more individual components of the subject drug delivery systems or devices can be located within a single housing and formed into a single unit. In some embodiments, one or more components of the subject systems or devices can be located in a first housing component, and one or more additional components of the subject systems or devices can be located in a second housing component, and the first and second housing components can be operably coupled to one another to form a single unit.

In some embodiments, a housing comprises one or more transparent or semitransparent windows that are made of a material that is at least partially transparent to light and is configured to allow ambient light to pass through the housing to reach a light sensor positioned therein. In some embodiments, a housing comprises one or more windows or openings that allow one or more components of the systems or devices to physically pass through.

### Data Management Component

Aspects of the disclosure include a data management component that is configured or adapted to communicate with the subject systems or devices and/or a user, e.g., to receive a report comprising a drug dose completion signal from a subject system or device, to send one or more commands to a subject system or device, or to send a reminder to a user that a drug dose is due to be administered at a certain time. In some embodiments, a data management component comprises a computer (e.g., a personal computer, a networked computer or a network server). In some embodiments, a data management device comprises a mobile computing device (e.g., a smart phone, or a laptop computer). In some embodiments, a data management component is an Internet-enabled device that is capable of sending and receiving information over the Internet. In some embodiments, a data management component comprises an application that is configured to manage one or more aspects relating to administration of a drug to a user (e.g., to record administration of individual drug doses to a patient, to remind a patient regarding upcoming drug dose administrations, to validate one or more drug identification characteristics by interacting with a remote database, etc.). In some embodiments, a data management component is configured to indicate to a user that one or more communication components are operational and/or are connected to one or more additional components of the subject drug delivery systems or devices. For example, in some embodiments, a data management component is configured to indicate to a user that the data management component is connected (e.g., via a Bluetooth or Wi-Fi connection) to a subject drug delivery system or device. In some embodiments, one or more indicator components on a subject drug delivery system or device, as described above, can further be used to indicate to a user that the data management component is connected to the system or device. Any suitable combination of indicator components on the data management component and/or the other components of the system or device can be used to indicate a connection status of the data management component to a user (e.g., connected, attempting to connect, not connected, disconnected, etc.).

In some embodiments, a data management component is configured or adapted to receive a report from a subject drug delivery system or device, and to record one or more aspects of the report for purposes of maintaining a patient's medical record/history. For example, in some embodiments, a data management component is configured to receive a report from a system or device that indicates a drug dose was administered to the patient, and the data management component records administration of the drug dose, including the date and time at which the drug dose was delivered. In some embodiments, a report can contain additional information relating to, e.g., the drug that was administered or the patient that received the drug. In some embodiments, a report can contain information relating to one or more operational states of the subject systems or devices. For example, in some embodiments, a report comprises information relating to, e.g., the temperature or temperature history of a system or device. In some embodiments, a report comprises information relating to a geographical location of the drug delivery system at the time of administration.

In some embodiments, a data management component is configured or adapted to receive one or more data inputs from a subject system or device, and to validate the one or more data inputs prior to proceeding with administration of the drug to the patient. For example, in some embodiments, a data management component is configured to receive a drug identification characteristic from a subject system or device and to verify that the drug identification characteristic is valid before proceeding with administration of the drug to the patient. In some embodiments, a data management component is configured to transmit one or more drug identification characteristics over the Internet to a remote database, and to receive an authentication signal in response, prior to administering the drug to the patient.

In some embodiments, a data management component is configured or adapted to receive one or more data inputs from a subject system of device, and to analyze the received data to determine whether a delivery stroke has been completed. For example, in some embodiments, a subject system or device is configured or adapted to transmit data from a deflection component and/or a sensor to a data management component, and to analyze the received data and compare the received data to one or more stored delivery signature parameters (e.g., a reference delivery signature) to determine whether the received data corresponds to a delivery signature for the device.

In some embodiments, a data management component is configured or adapted to determine whether a specific drug delivery system or device, or a component thereof is the result of an authorized sale from a manufacturer, and/or an authorized prescription of the drug from a prescribing health care provider (e.g., from a prescribing physician), in a specific geographical location (e.g., in a specific country). For example, in some embodiments, a data management component is configured to receive one or more drug identification characteristics from a subject drug delivery system or device (or a component thereof, e.g., a drug carousel or cartridge), and to transmit the one or more drug identification characteristics to a remote database. In some embodiments, a data management component is further configured or adapted to transmit a geographical location of the drug delivery system or device to the remote database as well. In some embodiments, a remote database is configured or adapted to compare the one or more drug identification characteristics with the geographical location received from the data management component to determine whether a specific drug delivery system or device, or component thereof (e.g., a drug capsule carousel or cartridge), is being used in the geographical location (e.g., the specific country) where it was sold.

In some embodiments, a data management component is configured to validate one or more operational states of the subject systems or devices prior to administration of the drug to the patient. For example, in one embodiment, a data management component is configured to determine whether a drug capsule is at a temperature that falls within a predetermined acceptable temperature range prior to administering the drug to the patient. In some embodiments, a data management component is configured to verify that a subject system or device is in a “ready” operational state prior to administering the drug to the patient.

Data management components in accordance with embodiments of the disclosure are configured to determine a date and time at which a drug is administered to a patient (e.g., a time stamp for the drug dose administration). In some embodiments, a data management component is configured to receive a drug dose completion signal from a subject system or device and is configured to determine the exact time of the drug administration based on additional information transmitted from the system or device. For example, in some situations, a subject system or device may not be operatively connected to a data management component at the specific date and time at which administration of the drug was carried out. In such instances, a subject system or device is configured to determine an elapsed time since completion of the drug administration procedure. When the system or device becomes connected to the data management component, a drug dose delivery signal as well as the elapsed time since the administration is transmitted to the data management component. The data management component then utilizes the transmitted information to back-calculate the specific date and time at which the drug administration procedure was completed and records this information in the patient's records.

### Controller, Processor, and Computer-Readable Media

In some embodiments, a subject system or device can comprise a controller, a processor, and a computer readable medium that are configured or adapted to control or operate one or more components of the subject systems or devices. In some embodiments, a system or device includes a controller that is in communication with one or more components of the subject systems of devices and is configured to control aspects of the systems or devices and/or execute one or more operations or functions of the subject systems or devices. In some embodiments, a system or device includes a processor and a computer-readable medium, which can include memory media and/or storage media. Applications and/or operating systems embodied as computer-readable instructions on the computer-readable memory can be executed by the processor to provide some or all of the functionalities described herein.

In some embodiments, a subject system or device includes a user interface, such as a graphical user interface (GUI), that is configured or adapted to receive an input from a user, and to execute one or more of the methods as described further herein. In some embodiments, a GUI is configured to display data or information to a user.

### Energy Harvesting

Aspects of the disclosure include energy harvesting systems coupled to a drug delivery device as disclosed herein. The use of an energy harvesting system allows the drug delivery device to harvest energy from its surrounding to power on-board communication electronics. This reduces or eliminates the need for a battery located on the device. In turn, this reduces challenges associated with environmentally friendly disposal of the devices, which are often disposable type devices. In some embodiments, mechanical energy is stored in a spring or similar means and this energy is recaptured at the time of use, such as at the end of inhalation or drug delivery cycle. In some embodiments, the spring or other storage mechanism supplies a generator with mechanical energy that is converted to electrical energy, rectified and regulated to power a wireless transmission from the drug delivery device to a mobile device or home-based receiver or hub. As previously described, this wireless transmission can provide drug delivery dose completed confirmation via smart device technology.

Various connection methods may be employed to wirelessly connect the self-powered drug delivery device to a user's smartphone. These may be segmented into direct and indirect methods:

Direct Methods—Communication Requiring No External Peripherals
BLE—Master/slave
BLE—Non-connectable connection (Advertising)
BLE—Scan response
WiFi
NFC (Near-field communication)
Cellular (existing logistics)
Audio (utilizing ultrasound identifiers)

Indirect Methods—Communication Requiring External Peripherals/Infrastructure
Internet (web-hosted)
‘Home hub’—RF (local)
‘Home hub’—Backscatter
‘Remote hub’—LORAWAN, SIGFOX, Narrowband Internet of Things (IoT)

In some embodiments of energy harvesting, BLE (Bluetooth Low Energy) “non-connectable” may be chosen due to it being a widespread and mature technology. With this approach, a connection does not need to be initiated between the device and phone, thereby reducing power consumption. In some embodiments, at least 1.04mJ is required to be delivered to a BLE module to perform the types of communications described herein. Because of energy losses between a power generating device and a BLE module, in some embodiments it is desirable to have a generator output of approximately 10mJ.

According to aspects of the disclosure, the energy harvesting source may comprise:
Ambient radiation
Fluid flow
Photovoltaic
Piezoelectric
Pyroelectric
Thermoelectric
Electrostatic
Magnetic inductive
Chemical

In the magnetic inductive category, the energy harvesting source may be of one of the following design configurations:
Impulse Energy Harvester
Levitating Magnetic Harvester
Cantilever Beam Harvester
Axial Flux Generator
Claw-Pole Microgenerator

Further details of the above-described “smart device” features may be found in co-pending published U.S. applications 2019/0321555 and 2021/0046247, incorporated herein by reference.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about”, “approximately” or “generally” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A handheld multi-unit dose dry powder inhaler device comprising:
a housing body having a swirl chamber therein, the swirl chamber being configured and arranged to allow a medicine capsule sufficient space to spin within the swirl chamber;
at least one air inlet fluidically connecting the swirl chamber to an aperture on an exterior surface of the housing body;
a mouthpiece coupled to the housing body and having an opening fluidically connected to the swirl chamber, the opening being configured to allow a user to draw air through the opening, thereby allowing an airstream to be drawn through the at least one air inlet, into the swirl chamber where it enables the capsule to spin and eject its contents into the airstream, and through the mouthpiece opening to deliver the contents into the user's respiratory system;
a rotatable downfall wheel having at least two capsule spaces that are each configured to hold a capsule, each of the capsule spaces having a center ramp located on a lagging side of the capsule space;
a drive mechanism configured to repeatably index the downfall wheel in a predefined angular increment such that one of the capsule spaces moves adjacent to the swirl chamber each time the downfall wheel is indexed; and
a pair of side ramps adjacent to the swirl chamber and the downfall wheel, the side ramps configured to cooperate with each of the center ramps to drive a capsule radially outward from the capsule space and into the swirl chamber, thereby enabling the device to automatically load multiple capsules one at a time sequentially into the swirl chamber.

2. The inhaler device of claim 1, wherein the device further comprises a rotatable carousel adjacent to the downfall wheel, the carousel being configured to rotate in a direction opposite to that of the downfall wheel, the carousel having at least three capsule spaces each configured to be preloaded with a capsule, the carousel and the downfall wheel being configured to cooperate to transfer one capsule from the carousel to the downfall wheel each time the carousel and the downfall wheel are indexed together.

3. The inhaler device of claim 2, wherein the downfall wheel rotates 90 degrees and the carousel rotates 22.5 degrees each time they are indexed together.

4. The inhaler device of claim 2, wherein the downfall wheel is provided with four capsule spaces, two of which are preloaded with capsules, and the carousel is preloaded with 28 capsules before first use.

5. The inhaler device of claim 4, wherein 30 capsules have been loaded into the carousel and two the 30 capsules are then advanced from the carousel into the two capsule spaces of the downfall wheel before the first use.

6. The inhaler device of claim 1, wherein the device further comprises a piercing hub provided with at least one piercing pin attached thereto, the piercing hub being configured to rotate the at least one piercing pin into a capsule while the downfall wheel is being indexed, thereby piercing the capsule while the capsule is moving.

7. The inhaler device of claim 6, wherein the piercing hub and the at least one piercing pin are configured to pierce a capsule perpendicular to a longitudinal axis of the capsule.

8. The inhaler device of claim 7, wherein the piercing hub and the at least one piercing pin are configured to pierce into the capsule on at least one of its hemispherical ends.

9. (canceled)

10. The inhaler device of claim 1, wherein the downfall wheel is configured to load capsules into the swirl chamber through a bottom opening of the swirl chamber, and wherein the downfall wheel is provided with a solid surface on its outer circumference between each of the capsule spaces, each solid surface being configured to close off the bottom opening of the swirl chamber in between index movements of the downfall wheel.

11. The inhaler device of claim 1, wherein the device further comprises a dose wheel that is indexed each time the downfall wheel is indexed, the dose wheel having a series of numbers located thereon, each of the numbers representing a capsule preloaded into the device, the device further comprising a window configured to show one of the numbers at a time to a user of the device indicating how many capsules remain in the device.

12. (canceled)

13. The inhaler device of claim 11, wherein the dose wheel further comprises a spiral groove configured to drive a slidable window in a radial direction relative to the dose wheel and wherein the series of numbers is arranged in a spiral pattern.

14. The inhaler device of claim 1, wherein the mouthpiece is configured to pivot between a closed position in which it can captivate a capsule in the swirl chamber and an open position in which it allows a capsule to be removed from the swirl chamber.

15. (canceled)

16. (canceled)

17. The inhaler device of claim 16, wherein the device further comprises a latch arm which is movable between a first position in which the mouthpiece cover can be retained in the closed position, thereby priming the device for its next cycle, and a second position in which the mouthpiece cover cannot be retained in the closed position and will not activate the next cycle.

18. (canceled)

19. A method of operating a multi-unit dose dry powder inhaler device, the method comprising the steps of:
providing an inhaler device having a swirl chamber, a mouthpiece, a mouthpiece cover and a predetermined number of capsules preloaded into the inhaler device;
pivoting the mouthpiece cover from a closed position to an open position, thereby uncovering the mouthpiece;
automatically piercing one of the preloaded capsules;
automatically indexing the pierced capsule into the swirl chamber; and
drawing an airstream through the mouthpiece, causing the capsule to spin and release its dry powder contents into the airstream.

20. The method of claim 19, wherein the automatic piercing and the automatic indexing are driven by the pivoting of the mouthpiece cover.

21. The method of claim 19, further comprising pivoting the mouthpiece open and discarding the capsule from the swirl chamber.

22. The method of claim 21, further comprising pivoting the mouthpiece closed and pivoting the mouthpiece cover from the open position to the closed position, wherein the mouthpiece cover is automatically returned to the open position unless the pivoting open the mouthpiece step has been performed first.

23. The method of claim 19, further comprising advancing a dose wheel when the mouthpiece cover is pivoted into the open position, the dose wheel showing a number to a device user indicating how many capsules remain unused in the device.

24. The method of claim 1, wherein the predetermined number of capsules preloaded into the inhaler device is at least three, and each of the method steps except the providing step are performed at least three times.

25. The method of claim 1, wherein the predetermined number of capsules preloaded into the inhaler device is at least thirty, and each of the method steps except the providing step are performed at least thirty times.
